(19) United States
(12) Patent Application Publication
(10) Pub. No.: US 2003/0162803 A1
(43) Pub. Date: Aug. 28, 2003

(54) Anhydrovinblastine for the treatment of cancer

(76) Inventors: Bruce Schmidt, Vancouver (CA); James Kutney, Vancouver (CA); Lawrence Mayer, North Vancouver (CA)

Correspondence Address: ELMAN TECHNOLOGY LAW, P.C., P O BOX 209, SWARTHMORE, PA, 19081-0209, US

(21) Appl. No.: 10/295,213

(22) Filed: Nov. 15, 2002

Related U.S. Application Data
(63) Continuation-in-part of application No. 09/946,149, filed on Sep. 4, 2001, now abandoned.
Application No. 09/946,149 is a continuation of application No. 09/380,540, filed on Jan. 21, 2000, now Pat. No. 6,326,376.
Application No. 09/380,540 is a 371 of international application No. PCT/CA98/00195, filed on Mar. 4, 1998.

(30) Foreign Application Priority Data
Mar. 4, 1997 (CA) 2199065
May 14, 1997 (CA) 2205314
Oct. 24, 1997 (CA) 2219095

(51) Int. Cl. A61K 31/4745

(52) U.S. Cl. 514/283

(57) ABSTRACT

The present invention is particularly directed to the use of a derivative of vinblastine, 3′,4′-dehydrovinblastine (3′,4′-anhydrovinblastine: AHVB), which differs from vinblastine in that it possesses a double bond at the 3′,4′ position of the catharanthine nucleus rather than the hydroxyl group that is present in the parent structure, in the treatment of cancer.

### CROSS REFERENCES TO RELATED APPLICATIONS

This application is a continuation-in-part of Ser. No. 09/946,149, which is a continuation of Ser. No. 09/380,540, filed Mar. 4, 1998, now U.S. Pat. No. 6,326,376, which is anational stage of PCT/CA98/00195, filed Mar. 4, 1998. The aforesaid PCT application claims priority from Canadian Patent Application 2,199,065, filed Mar. 4, 1997; and Canadian Patent Application 2,205,314, filed May 14, 1997; and Canadian Patent Application 2,219,095 filed Oct. 24, 1997. The entire contents of all of the aforementioned applications are hereby incorporated herein by reference.

### TECHNICAL FIELD

The present invention is related generally to the use of antineoplastic vinca alkaloids as antitumor agents. More particularly, the present invention is related to providing use for a derivative of vinblastine, anhydrovinblastine (hereinafter AHVB), as an antineoplastic agent with improved therapeutic properties, demonstrating a significantly higher maximum tolerated dose and less toxicity than its parent and related compounds.

### BACKGROUND OF INVENTION

Due to a high degree of unpredictability, classic techniques of drug development are inventive. Mostly through a process of elimination, a large number of natural products and synthetic chemical compounds are screened for desired effects, using a series of increasingly complex systems, beginning with simple in vitro cell-level assays, progressing to animals and finally human clinical trials. But, due to essential characteristics such as adsorption, distribution and metabolism, the initial in vitro tests that can not take these features into account could eliminate a powerful drug that does not perform well in such simple systems. The drug could be metabolized to different compounds in animal models than in humans, which may also demonstrate different adsorption or distribution patterns. Or finally, compounds can look very promising all the way through clinical trials, but then demonstrate unpleasant side effects or a high degree of tolerance when used by the human population at large. It is never obvious which compound will continue to look promising as each stage of tests and development are initiated.

Control of tumorous growth has been achieved to a certain degree using oncolytic vinca alkaloids as antitumor agents alone or in combination with other antineoplastic drugs in cancer chemotherapy for more than 20 years. Approximately 30 alkaloids with a wide range of pharmacological activities have been extracted from the Vinca rosea (Catharanthus roseus), commonly known as the periwinkle plant. Of these, only vinleurosine, vinrosidine, vinblastine and vincristine possess significant anti-tumor activity. In particular, vinblastine and vincristine have been used widely as single agents and in combination with outer antineoplastic drugs in cancer chemotherapy. In addition to the naturally occurring alkaloids, some vinca alkaloid analogues have been synthesized by functional transformation or by semisynthetic processes (R. J. Cersosimo, et al., Pharmacotherapy 3:359-274, 1983; P. Mangency, et al., Org. Chem. 44:3765-3768, 1979; R. Maral, et al., Cancer Lett. 22:49-54, 1984).

Chemically, these vinca alkaloids have a dimeric asymmetric structure composed of 2 nuclei linked by a carbon-carbon bond; a dihydroindole nucleus (vindoline), which is the major alkaloid contained in the periwinkle, and the indole nucleus catharanthine (FIG. 1). The structural difference between vincristine and vinblastine exists at the R1 position while vinblastine and vindesine differ with regard to the R2 and R3 substituents.

The mode of action of the antineoplastic vinca alkaloids has yet to be completely understood. However, it has been established that the antitumor activity is directly related to the high binding affinity of these compounds for tubulin, the basic protein subunit of microtubules (R. A. Bender and B. Chabner, In: Chabner (ed) Pharmacol. Princ. of Cancer Treat., Saunders, Phil, PA, p. 256, 1982; W. A. Creasey, In: Hahn (ed) Antibiotica, Vol. 2, Springer, Berlin, p. 414, 1979). The consensus is that these agents arrest cell mitosis at metaphase by preventing tubulin polymerization to form microtubules and by inducing depolymerization (R. J. Owellen and C. A. Hartke, Cancer Res., 36:1499-1504, 1976; R. H. Himes and R. N. Kersey, Cancer Res., 36:3798-3806, 1976; R. S. Camplejohn, Cell Tissue Kinet. 13:327-332, 1980). As such, the vinca alkaloids are cell cycle-specific anti-mitotic agents, or spindle poisons. The binding affinity of the vinca alkaloids to tubulin correlates poorly with the relative ability of vincristine, vinblastine and vindesine to inhibit cell growth (R. S. Camplejohn, supra; P. J. Ferguson and C. E. Cass, Cancer Res., 45:5480-5488, 1985). The major difference in anti-tumor activity between these drugs appears, therefore, to relate to their retention in tumor tissue (P. Ferguson, supra; J. K. Horton et al., Biochem. Pharmacol. 37:3995-4000, 1988). In a similar vein, the different toxicity profiles of the vinca alkaloids seems related to tissue uptake and retention properties rather than to inherent tubulin binding affinity. For example, studies have demonstrated that vincristine is more potent than vinblastine or vindesine in blocking fast axoplasmic transport in nerve cells (S. Ochs and R. Worth, Proc. Am. Assoc. Cancer Res., 16:70, 1975; S. Y. Chan et al., J. Neurobiol. 11:251-264, 1980). In addition, it is taken up into nerves 4 times faster than the other drugs (Z. Iqbal and S. Ochs, J. Neurobiol., 11:251-264, 1980) and exhibits an extended terminal elimination phase of plasma clearance, suggesting a more prolonged exposure to vincristine than to the other vinca alkaloids (R. L. Nelson et al., Cancer Treat. Rev., 7:17-24, 1980).

The in vitro and in vivo differences observed between the vinca alkaloids are striking given the subtle chemical alterations displayed by the various agents relative to their large, complex molecular structure. For example, vincristine is very effective in treating human rhabdosarcomas transplanted in nude mice whereas vinblastine is not active in this system (N. Bruchovsky et al., Cancer Res. 25:1232-1238, 1965). This difference is obtained simply as a result of the substitution of an aldehyde group for a methyl group at the R1 position. Further, this chemical substitution leads to a shift in the toxicology profile such that peripheral neuropathy (in the absence of hematological toxicity) is dose limiting in humans for vincristine whereas anemia and leucopenia are typically dose limiting for vinblastine (W. P. Brads, Proc. Int. Vincaalkaloid Symposium, 95-123, 1980; S. S. Legha, Med. Toxicol., 1:421-427, 1986). A particularly interesting therapeutic profile has been observed for a new semisynthetic vinca alkaloid named Navelbine™ (vinorelbine, 5′-noranhydroblastine). This compound is less potent than vinblastine and vincristine against murine P388 and L1210 leukaemia but is active against cells derived from human lung cancer whereas the other vinca alkaloids are inactive (S. Cros, et al., Seminars in Oncology, 16:15-20, 1989). As well, clinical trials on Navelbine™ support its utility in treating non-small cell lung cancer (A. Depierre et al., Am. J. Clin. Oncol., 14:155-119, 1991; A. Yokoyama et al., Am. Soc. Clin. Oncol., 11:957, 1992). The toxicity profile of this agent appears similar to that of vinblastine, where hematological toxicities and not neurological side effects are dose limiting.

Vincristine has proved particularly useful as an intravenously administered oncolytic agent in combination with other oncolytic agents for the treatment of various cancers including central-nervous-system leukaemia, Hodgkin's disease, lymphosarcoma, reticulum-cell sarcoma, rhabdomyosarcoma, neuroblastoma, and Wilma tumor. It is for intravenous (IV) use only and the intrathecal administration is uniformly fatal. Following single weekly doses, the most common adverse reaction is hair loss; the most troublesome are neuromuscular in origin. When single weekly doses of the drug are employed, the adverse reactions of leukopenia, neuritic pain, constipation, and difficulty in walking can occur. Other adverse reactions that have been reported are abdominal cramps, ataxia, foot drop, weight loss, optic atrophy with blindness, transient cortical blindness, fever, cranial nerve manifestations, parehesia and numbness of the digits, polyuria, dysuria, oral ulceration, headache, vomiting, diarrhoea, and intestinal necrosis and/or perforation.

Navelbine™ (vinorelbine tartrate) is a novel vinca alkaloid in which the catheranthine unit is the site of structural modification. Its anti-tumor activity is also thought to be due primarily to its ability to interfere with microtubule activity thereby inhibiting mitosis at metaphase through its interaction with tubulin. It is indicated in the treatment of advanced non-small cell lung cancer as a single agent or in combination, administered by intravenous route only. Its side effects include phlebitia or extravasion injury as it is a moderate vasicant. Studies on adverse reactions based on use of Navelbine™ as a single agent indicate Granculocytopenia as the major dose-limiting toxicity, although it was generally reversible and not cumulative over time. Mild to moderate peripheral neuropathy manifested by pareathesia and hypesthesia are the most frequently reported neurologic toxicities, occurring in 10% of patients. Mild to moderate nausea occurs in roughly one-third of patients treated with Navelbine™ with a slightly lesser fraction experiencing constipation, vomiting, diarrhoea, anorexia, and stomatitis.

Compounds exhibiting lessened toxic effects with equal or greater chemotherapeutic activity remain to be achieved. Thus, a need remains for a drug providing improved anti-tumor efficacy for the treatment of cancer.

It is, therefore, an object of the present invention to provide a method of treating cancer which comprises administering to a human patient suffering from cancer and in need of treatment, an amount of AHVB, effective to arrest or significantly slow the progress of the disease.

It is another object of the present invention to provide a method of using AHVB as an antitumor agent, comprising therapeutic amount of the chemical substance of the present invention to arrest tumorous growth.

The above and various other objects and advantages of the present invention are achieved by administration of a derivative of vinblastine, AHVB. Other objects and advantages will become evident from the following detailed description of the present invention.

### SUMMARY OF INVENTION

The present invention is particularly directed to the use of a derivative of vinblastine, 3′,4′-anhydrovinblastine (AHVB), which differs from vinblastine in that it possesses a double bond at the 3′,4′ position of the caranthine nucleus rather than the hydroxyl group that is present in the parent structure, as an antineoplastic agent in the therapeutic treatment of cancer.

In accordance with an aspect of the present invention, there is provided a use of 3′,4′-anhydrovinblastine, or variants thereof, as an antineoplastic agent in the treatment of cancer.

In accordance with another aspect of the present invention, there is provided a method of treating cancer in a mammal comprising administering to said mammal an effective amount of 3′,4′-anhydrovinblastine or a pharmaceutically acceptable salt thereof, wherein said cancer is an advanced cancer.

In accordance with another aspect of the present invention, there is provided a use of 3′,4′-anhydrovinblastine as an antineoplastic agent in the treatment of cancer, wherein the concentration of 3′,4′-anhydrovinblastine is at significantly higher maximum concentration than therapeutically acceptable concentrations for vinblastine or Navelbine™ for use in the treatment of cancer.

In accordance with another aspect of the present invention, there is provided a method of treating cancer in a mammal comprising administering to said mammal an effective amount of 3′,4′-anhydrovinblastine (AHVB) or a pharmaceutically acceptable salt thereof, wherein said effective amount comprises a dose of between about 2 and about 30 mg AHVB/m².

In accordance with another aspect of the present invention, there is provided a use of 3′,4′-anhydrovinblastine as an antineoplastic agent in the treatment of cervical cancer.

In accordance with another aspect of the present invention, there is provided a use of 3′,4′-anhydrovinblastine as an antineoplastic agent in the treatment of lung cancer.

In accordance with another aspect of the present invention, there is provided a pharmaceutical composition comprising 3′,4′-anhydrovinblastine (AHVB) and one or more pharmaceutically acceptable, inert or physiologically active carriers, diluents or adjuvants, wherein said pharmaceutical composition is formulated for administration to a mammal at a dose of between about 2 and about 30 mg AHVB/m².

### BRIEF DESCRIPTION OF THE DRAWINGS

FIG. 1 depicts the chemical structure of some vinca alkaloids.

FIG. 2 depicts comparison of effects of administering a single intraperitoneal injection, at a subacutely toxic dose, of vincristine, Navelbine™ and AHVB to Nb rats bearing single well-developed, subcutaneous Nb2-U17 tumor transplants on average tumor weight and average weight of the rat as a function of time.

FIG. 3 depicts comparison of the effects of administering a single intraperitoneal injection, at a half subacutely toxic dose of vincristine, Navelbine™ and AHVB to Nb rats bearing single well-developed, subcutaneous Nb2-U17 tumor transplants on average tumor weight and average weight of the rat as a function of time.

FIG. 4 depicts changes in mean animal weight of BDF1 mice bearing intraperitoneal P388 tumors following IV administration of saline, vincristine, Navelbine™ and AHVB.

FIG. 5 depicts an example cytotoxicity curve used to estimate the IC₅₀ of various vinca alkaloids.

FIG. 6 depicts P388 anti-tumor activity of selected formulations of vinca alkaloids.

FIG. 7 depicts a dose response curve obtained for AHVB when used to treat BDF1 mice bearing P388 tumors.

FIG. 8 depicts cytotoxicity curves used to estimate the IC₅₀ of AHVB on the cell lines SKOV3 and C-4.

FIG. 9 depicts mean tumor weight in grams over time (30 days period) following administration at days 1, 5, and 9, of Navelbine™, bisulphate AHVB, ditartrate AHVB, and control.

FIG. 10 depicts measured AHVB serum concentration over a 0 to 72 h time frame for individual patients in a Phase I clinical trial of AHVB.

FIG. 11 depicts the clearance of AHVB for patients in a Phase I clinical trial at their respective dose level.

FIG. 12 depicts the linear increase in AUC with Dose for patients enrolled in a Phase I clinical trial of AHVB.

FIG. 13 depicts the increase in Half Life with Dose for patients enrolled in a Phase I clinical trial of AHVB.

FIG. 14 depicts the linear increase in maximum plasma concentration (Cₘₐₓ) with Dose for patients enrolled in a Phase I clinical trial of AHVB.

FIG. 15 depicts a Goodness of Fit Plot demonstrating the ability of a two-compartment pharmokinetic model to predict the pharmokinetic properties of AHVB.

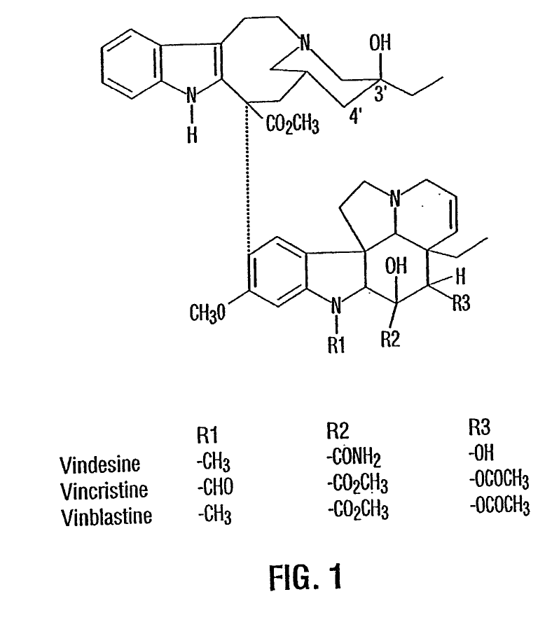

### DETAILED DESCRIPTION OF THE INVENTION

There are many possible derivatives or variations of vinblastine possible. However, there is no certainty, even to those skilled in the area of anti-cancer drug development, that any such derivatives will be as efficacious or even more efficacious than the parent compound. This takes much testing and experimentation.

The term “variants” for purposes of 3′,4′-anhydrovinblastine means any chemical structure that is a derivative of 3′,4′-anhydrovinblastine achieved through conservative substitution of side groups, yet still exhibits the same or similar antineoplastic properties as 3′,4′-anhydrovinblastine.

Examples of cancers which may be may be treated, stabilized, or prevented in accordance with the present invention include, but are not limited to leukaemia, carcinomas, adenocarcinomas, melanomas and sarcomas. Carcinomas, adenocarcinomas and sarcomas are also frequently referred to as “solid tumors,” examples of commonly occurring solid tumors include, but are not limited to, cancer of the brain, breast, cervix, colon, head and neck, kidney, lung, ovary, pancreas, prostate, stomach and uterus, non-small cell lung cancer and colorectal cancer.

The term “leukaemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “sarcoma” generally refers to a tumor which originates in connective tissue, such as muscle, bone, cartilage or fat, and is made up of a substance like embryonic connective tissue and is generally composed of closely packed cells embedded in a fibrillar or homogeneous substance. Sarcomas include soft tissue sarcomas, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. In one embodiment of the present invention, AHVB is used to treat a patient with a sarcoma. In another embodiment, the sarcoma is a soft tissue sarcoma. In other embodiments, the sarcoma is a metastatic sarcoma or a metastatic sarcoma to the lungs.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidernal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum. In one embodiment of the present invention, AHVB is used to treat a patient with a carcinoma.

The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia. Examples include, but are not limited to, adenocarcinomas of the breast, lung, pancreas and prostate. In one embodiment of the present invention, AHVB is used to treat a patient with an adenocarcinoma. In another embodiment, the adenocarcinoma is breast cancer. In other embodiments, the adenocarcinoma is prostate cancer or pancreatic cancer.

Additional cancers encompassed by the present invention include, for example, Hodgkin's Disease, Non-Hodgkin's lymphoma, multiple myeloma, neuroblastoma, rhabdomyosarcoma, primary thrombocytosis, primary macroglobulinemia, small-cell lung tumors, primary brain tumors, malignant pancreatic insulanoma, malignant carcinoid, urinary bladder cancer, premalignant skin lesions, gliomas, testicular cancer, thyroid cancer, esophageal cancer, genitourinary tract cancer, malignant hypercalcemia, endometrial cancer, adrenal cortical cancer, mesothelioma and medulloblastoma.

Characterization of AHVB Anti-Tumor Activity in vitro

Cytotoxicity experiments on AHVB were performed as direct comparisons with vincristine and Navelbine™ in order to assess its inherent antineoplastic profile against a variety of tumor cell types relative to other relevant vinca alkaloids. The cytotoxicity of AHVB was investigated in vitro against a panel of tumor cell lines of varying lineage in order to determine the specificity of its antitumor activity with respect to cell type. The tumor lines studied were P388 lymphocytic leukaemia (a murine lymphocytic leukaemia), Noble (Nb) rat U17 lymphoma, MCF7 human breast carcinoma, H460 human non-small cell lung carcinoma, K562 human erythroleukaemia and LS180 human colon carcinoma based on established NCI in vitro new anti-cancer drug cytotoxicity screening protocols.

Standard dose response cytotoxicity assays (R. Mosmass, J. Immunol. Meth., 65:55-64, 1983) were utilized to determine the IC50 (drug concentration required to induce 50% inhibition of tumor cell growth) for vincristine, Navelbine™ and AHVB. The results are presented in Table 1. The indicated cell lines were obtained from either the ATCC or NCI tumor repository and were cultured in tissue culture media by standard techniques well known to those skilled in the art, prior to dilution to a defined cell concentration required for the studies in 96 well plates.

A wide range of drug concentrations were exposed to tumor cells growing at log phase in 96-well microtitre plates. Cell concentrations depended on the cell line as well as the length of time to be cultured. Typically, P388 cells were plated at a concentration of 30,000, 2,000 and 750 cells per well for studies lasting 1, 3 and 7 days, respectively. MCF7 cells were plated at a concentration of 7,000 and 1,500 cells per well for studies lasting 3 and 7 days, respectively. H460 cells were plated at a concentration of 2,500 and 1,000 cells per well for studies lasting 3 and 7 days, respectively. K562 cells were plated at a concentration of 1,500 and 10,000 cells per well for studies lasting 1 and 3 days, respectively. LS180 cells were plated at a concentration of 5,000 and 20,00 cells per well for studies lasting 3 and 7 days, respectively. After plating all cell lines were incubated (CO₂ incubator at 37° C., 5% CO₂) for 24 hours prior to addition of the cytotoxic agent (See Table 1).

Subsequently the plates were incubated for the indicated time period. At specified times, cells were washed and subsequently exposed to the dye inclusion marker MTT (3-(4,5-dimethylthiazol-2-yl)-2,5-diphenyl tetrazolium), which accumulated into viable cells. MTT was added to the cells at a final concentration of 50 μg per well. After a 4 hour incubation, the cells were washed free of media and unreacted MTT, prior to addition of DMSO which was required to solubilize the insoluble formazan precipitate that formed in viable cells. After the sample was mixed through repeated pipetting, the coloured product was measured using a plate reader operating at 570 nm. The absorbance values obtained for cells cultured in the absence of drug was assumed to represent 100% viability. Experiments were repeated to substantiate any differences noted between AHVB and other vinca alkaloids.

Characterization of AHVB Antitumor Activity in vivo

Evaluation of in vitro cell cytotoxicity was followed by studies regarding the antineoplastic activities of AHVB in three in vivo rodent models. Thus, anti-tumor activity of AHVB was determined using a rat solid tumor model (U17 lymphoma), the murine P388 tumor model (R. Noble, et al., Cancer Res., 37:1455-1460, 1977; P. W. Gout et al., Biochem Cell Biol., 64:659-666, 1986), and a H460 SC Tumor mouse model.

The U17 cell line was originally derived from a transplantable malignant lymphoma that arose spontaneously in male Noble rats (British Columbia Cancer Research Centre Joint Animal Breeding Facility with parents obtained from the National Institutes of Health, Bethesda, Md.). The cell line is prolactin dependent and can readily be cultured in vitro. U17 derived solid tumors are generated by subcutaneous injection (via the trocar method) of a small (2 mm²) piece of tumor tissue obtained from male Noble rat. Tumor tissue used for the implants arose two weeks after injection of 5×10⁶ U17 cells (from culture) subcutaneously in the nape of the neck. For assessing the anti-tumor activity of AHVB, tumor bearing animals (2-4 gm tumors) were given a single treatment of drug and tumor size was measured as a function of time following treatment. The anti-tumor activity was assessed at a series of different doses in order to determine the maximum therapeutic dose of AHVB. Comparative studies between vincristine, vinblastine and AHVB were performed. For these studies anti-tumor activity was determined at the maximum therapeutic dose of each drug.

Antitumor studies on mice focussed, in one case, on the P388 leukaemia model. This is a standard NCI model for evaluation of new anti-cancer agents and it has been demonstrated to be sensitive to treatment with vinca alkaloids. This is an ascitic tumor model that was generated by intraperitoneal inoculation of 1×10⁶ P388 cells (derived from culture, with an original cell line obtained from the NCI tumor repository) in BDF1 mice (Charles Rivers). One day after tumor cell inoculation, mice were treated with a single intravenous injection of drug. Animal weight was monitored daily and tumor progression was measured as an increase in animal weight and through estimation of survival time. Therapy was described by a decrease in tumor progression and an increase in survival time relative to an untreated control group. Initial studies established the maximum therapeutic dose for AHVB. Subsequently comparative studies with vincristine and Navelbine™ were initiated where animals were treated with each drug at the maximum therapeutic dose.

The Canadian Council on Animal Care Guidelines were strictly adhered to and all animal protocols employed were approved by the Animal Care Committees of UBC and the BCCA. Animals were evaluated twice daily for any signs of stress (tumor or drug related) and if an animal appeared to be suffering (excessive weight loss or gain, lethargy, scruffy coat, etc.) than the animal was terminated.

Identification of Maximum Tolerated Dose of AHVB

Range-finding acute (14 day), single dose toxicity studies were performed in healthy male Nb rats in order to determine the maximum tolerated dose of vincristine sulfate, Navelbine T and AHVB when administered as a single, intraperitoneal injection in these rodents (see Table 2).

To this end, healthy non-tumor bearing male Nb rats (weight range 333-399 grams) were divided in groups of 3 animals. Each group was used to test one drug at one dosage. In a group, each animal received one intraperitoneal injection at a particular dose, as indicated in Table 2. The volumes within which the drugs were administered depended on the concentration of the drug solution (in saline) and the weight of the animals, and ranged from 0.1-1.0 ml. Saline was used as a control. The highest dose of each drug which allowed survival of all animals in a group (3 out of 3) was taken as the subacutely toxic dosage for the drug, i.e. 0.7 mg/kg for vincristine, 2.0 mg/kg for Navelbine™ and 3.0 mg/kg for AHVB.

The health of the animals was assessed by daily weight measurements in addition to behavioural indications of stress. Animals continued to be monitored throughout the complete 14 day study period. Animals were euthanized in the event of signs of severe stress or weight loss in excess of 20%. All animals were necropsied at the end of the study period or at the time of premature euthanasia. Once weight loss in excess of 20% or premature animal death was noted at a dose level, the dose was decreased until the weight loss nadir was less than 20% and no premature animal deaths were observed.

Studies in the Rat U17 Lymphoma Model

Cultures of the non-metastatic, pre-T Nb2 lymphoma line originally developed at The University of British Columbia and designated Nb2-U17 (Anticancer Research 14:2485-2492, 1994), and are available from the British Columbia Cancer Research Centre. Cells from exponentially growing Nb2-U17 suspension cultures were injected subcutaneously into methoxyflurane-anaesthetised, mature male Nb rats (5 rats; 310-380 grams; 5×10⁶ cells/rat in 1 ml of culture medium) at the nape of the neck using a 1.5″ 20-gauge needle. At about 3 weeks, when the tumors reached a size of 4-7 cm (length+width), the animals were sacrificed and the tumors used for transplantation as described below.

A tumor from a rat was excised, minced and the tumor tissue was put into trocars (2″, 13 gauge). The tissue samples were implanted subcutaneously in the nape of the neck of methoxyflurane-anaesthetised male Nb rats (248-404 grams; 1 trocar per rat). This procedure was repeated 5 times to get a total of 60 tumor-bearing rats to be used for efficacy studies of the 3 drugs.

When the tumors were well established (1.5-2 weeks later), three separate groups of 20 rats, as closely matched as possible in terms of both tumor weight and rat weight, were selected for administration of the three test articles (i.e. one group for each test article).

Vincristine was administered to rats weighing 281-384 grams, bearing tumors weighing 6.3-16.3 grams. Navelbine™ was administered to rats weighing 274-389 grams bearing tumors weighing 9.1-23.3 grams. AHVB was administered to rats weighing 303-400 grams, bearing tumors weighing 7.9-25.9 grams. Tumor weights were estimated using the hemi-ellipsoid model (weight in grams=length×depth×π/6 in cm).

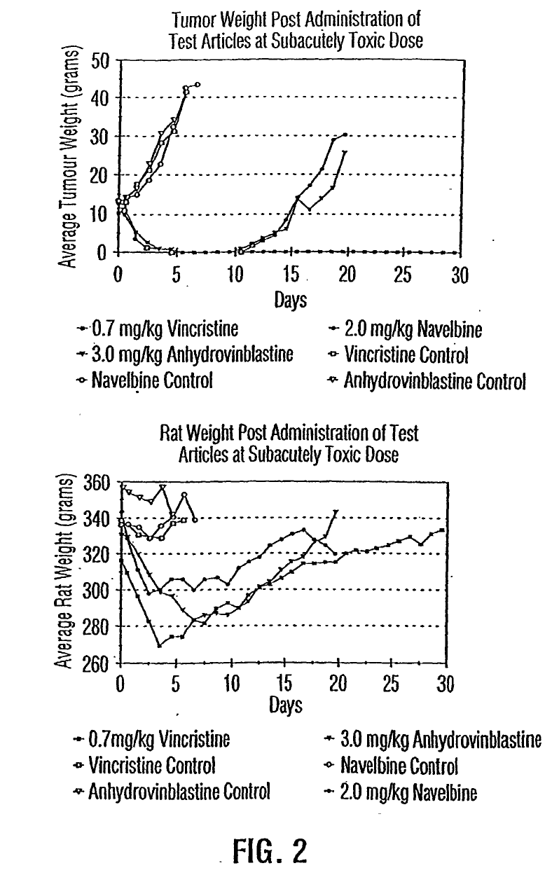

The oncolytic effects of each of the three drugs were assessed at a subacutely toxic dose, determined for each drug in preliminary studies using non-tumor-bearing, mature male Nb rats, i.e. 3.0, 2.0 and 0.7 mg/kg for AHVB, Navelbine™ and vincristine, respectively as illustrated in FIG. 2. In addition, each drug was assessed at 50% and 25% of its subacutely toxic dose. Five tumor-bearing rats were used to evaluate the effect at each dose level. The drugs were administered intraperitoneally as a single bolus in a volume of 0.19-0.31 ml, as indicated by the weight of the animals. To this end, drug preparations were diluted to appropriate concentrations using sparged saline adjusted with acetic acid to pH 4.2. For each drug, a group of 5 control rats received an intraperitoneal injection of the equivalent amount of saline (pH 4.2). The tumor-bearing rats were organized in the following groups:

| Group | Drug/Saline | Dose(mg/kg) |
|---|---|---|
| 1 | saline | — |
| 2 | AHVB | 3.0 |
| 3 | AHVB | 1.5 |
| 4 | AHVB | 0.75 |
| 5 | saline | — |
| 6 | Navelbine ™ | 2.0 |
| 7 | Navelbine ™ | 1.0 |
| 8 | Navelbine ™ | 0.5 |
| 9 | saline | — |
| 10 | vincristine | 0.7 |
| 11 | vincristine | 0.35 |
| 12 | vincristine | 0.175 |

Following administration of the test articles, the weight and tumor size (using calipers) of each animal was determined daily until the tumor reached an estimated weight of 35 grams, or started to ulcerate, at which times the animals were sacrificed (by carbon dioxide inhalation) and subjected to necropsy. Animals were also monitored at least daily for signs of stress for the full length of the study. Animals manifesting severe symptoms of stress (rapid weight loss, panting, hunched posture, scruffy coat) were also sacrificed and a necropsy performed.

Anhydrovinblastine Sulfate (3′,4′-dehydrovinblastine) was obtained from the British Columbia Cancer Agency (BCCA), Investigational Drug Section. Vincristine Sulfate (Sulfate of 22-oxovincaleukoblastine) was obtained from David Bull Laboratories Ltd., Australia. Navelbine™ (vinorelbine tartrate; 3′,4′-didehydro-4′-deoxy-C′-norvincaleukoblastine-di-L-tartrate) was purchased from Burroughs Wellcome Inc., Canada; 0.9% Sodium Chloride Injection USP, pH 4.2 was purchased from Baxter.

The methodology involving animals was approved by the BCCA's Institutional Animal Care Committee (IACC) at UBC prior to conducting the studies (Animal Care Certificate No. A94-1602). During the study the care, housing and use of animals was performed in accordance with the Canadian Council on Animal Care Guidelines.

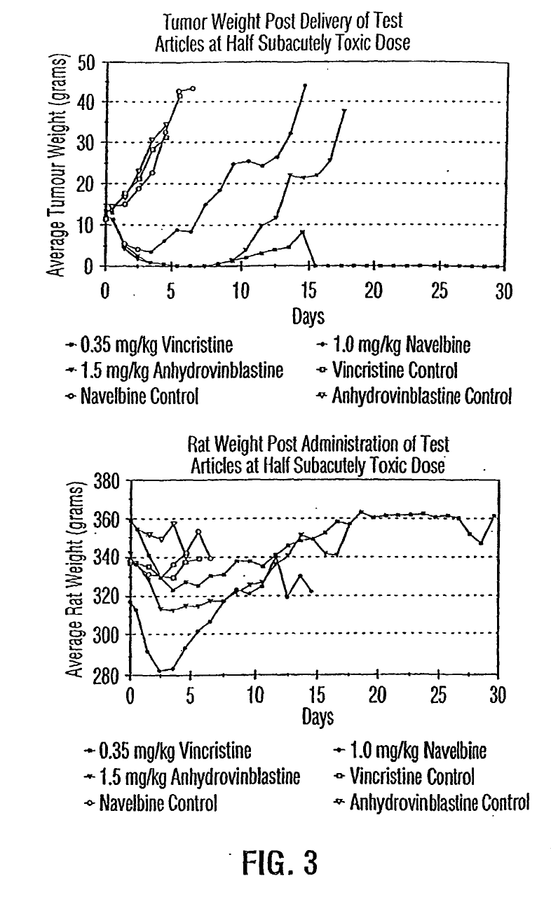

The results of the efficacy studies are given in FIGS. 2-3. FIGS. 2-3 present averages of data from 5 or fewer animals.

The effect of administering a single intraperitoneal, subacutely toxic dose of AHVB, Navelbine™ and vincristine on the size of single, well-established Nb2-U17 lymphoma transplants (average weight 10-13 grams) and the weight of the animals, as a function of time are demonstrated in FIG. 2. Whereas the tumors in the control animals continued to increase in size to an average weight of about 40 grams in 6 days, the tumors in the drug-treated animals in each case regressed to essential non-palpability within 5 days of drug administration. After day 10, recurrence of tumors in Navelbine™- and AHVB-treated animals occurred to about the same extent. In contrast, recurrence of tumors was not observed in vincristine-treated animals (not even on day 29). FIG. 2 also shows that the animals lost weight following drug administration. However, most of the weight was regained after about 17 days. As controls for each drug, Nb2-U17 tumor transplant-bearing rats injected with saline were used. For each of the six groups five animals were used. Vincristine sulfate (0.7 mg/kg) was administered in a volume of 0.20-0.23 ml to rats weighing 281-331 grams bearing tumors weighing 7.6-14.2 grams. Navelbine™ (2.0 mg/kg) was administered in a volume of 0.24-0.31 ml to rats weighing 297-389 grams bearing tumors weighing 11.5-13.7 grams. AHVB (3.0 mg/kg) was administered in a volume of 0.20-0.24 ml to rats weighing 314-374 grams bearing tumors weighing 8.2-14.2 grams. Vincristine sulfate controls: saline was administered in a volume of 0.21-0.26 ml to rats weighing 294-370 grams bearing tumors weighing 9.4-14.6 grams. Navelbine™ controls: saline was administered in a volume of 0.25-0.29 ml to rats weighing 310-365 grams bearing tumors weighing 9.5-18.2 grams. AHVB controls: saline was administered in a volume of 0.19-0.25 ml to rats weighing 303-400 grams bearing tumors weighing 7.9-16.6 grams. The efficacies of each drug were determined separately at three different dosages versus a control.

FIG. 3 shows the anti-tumor effects of the three drugs at 50% of their individual maximum tolerated doses. The data show that Navelbine™ was less potent than AHVB which in turn was less potent than vincristine.

Nb2-U17 tumor transplant-bearing rats injected with saline were used as controls. For each of the six groups five animals were used. Vincristine sulfate (0.35 mg/kg) was administered in a volume of 0.23-0.27 ml to rats weighing 327-384 grams bearing tumors weighing 6.4-13.4 grams. Navelbine™ (1.0 mg/kg) was administered in a volume of 0.24-0.28 ml to rats weighing 296-351 grams bearing tumors weighing 9.1-14.1 grams. AHVB (1.5 mg/kg) was administered in a volume of 0.20-0.23 ml to rats weighing 308-359 grams bearing tumors weighing 9.7-19.5 grams. Vincristine sulfate controls: saline was administered in a volume of 0.21-0.26 ml to rats weighing 294-370 grams bearing tumors weighing 9.4-14.6 grams Navelbine™ controls: saline was administered in a volume of 0.25-0.29 ml to rats weighing 310-365 grams bearing tumors weighing 9.5-18.2 grams. AHVB controls: saline was administered in a volume of 0.19-0.25 ml to rats weighing 303-400 grams bearing tumors weighing 7.9-16.6 grams. The efficacies of each drug were determined separately at three different dosages versus a control. In FIG. 3, results of the three drugs at equivalent, i.e. half subacutely toxic, dosages are compared. The controls in FIG. 3 are the same as in FIG. 2.

Studies in the Murine P388 Model

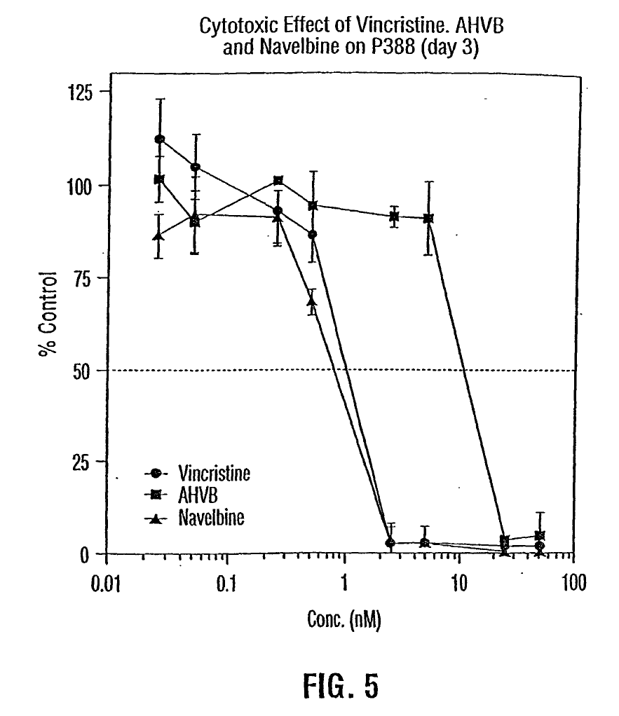

A cytotoxicity curve was generated to estimate the IC₅₀ of vincristine, Navelbine™ and AHVB in the murine P388 cell line (see FIG. 5). In this study, P388 cells derived from an ascitic tumor grown in BDF1 were first separated from red cells employing Ficoll-Paque. Isolated white cells were washed twice then placed in serum containing tissue culture media (1×10⁵ cells per ml of RPMI 1640 supplemented with L-glutamine, penicillin, streptomycin and 10% fetal bovine serum) and cultured for 2 hours. All non-adherent cells were collected and that cell population was defined as P388 cells and used for cytotoxicity assays 24 hours later. Cytotoxicity assays were performed as described in the section entitled Characterization of AHVB Anti-tumor Activity In Vitro. The drug concentrations used are indicated on the X-axis. Vincristine is represented by the filled circles, Navelbine™ by the filled triangles and AHVB by the filled squares.

The in vivo anti-tumor activity of AHVB was compared to that of vincristine, Navelbine™ in the BDF1-murine P388 model in the procedure as follows. P388 cells were derived from the ascities of previously injected female BDF1 mice (19-21 grams) P388 cells, from the NCI tumor repository were inoculated directly into mice. The cells arrive from NCI frozen in 1 ml aliquots. These samples were thawed rapidly at 37° C. and subsequently injected (within 1 hour) intraperitoneally into two mice, 0.5 ml per mouse. One week (7 days) after inoculation, the tumor cells were harvested by removing peritoneal fluid using a sterile syringe with a 22 gauge needle. The cells, pooled from two animals, were counted using a hemocytometer, diluted (RPMI media) to a concentration of 2×10⁶ cells/ml and 0.5 ml was then re-injected into each of two BDF1 mice. Remaining cells were washed and placed into a DMSO containing media and frozen (in freezer packs that cool at a defined rate). This process was repeated weekly over a 2-week period. Cells used for anti-tumor studies were collected from the third passage to the 20th passage. After the 20th passage the cells were no longer used for experimental studies. Newly established cells were derived from the frozen cells prepared as described above.

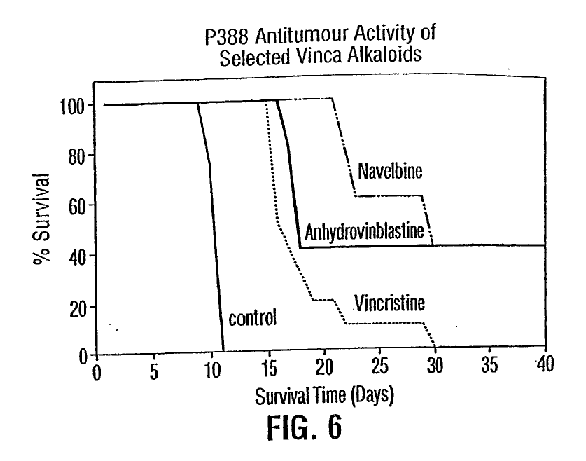

Groups (five mice per group) of female BDF1 mice (Charles Rivers, Canada) were injected (intraperitoneal) with 10⁶ P388 cells (as described above). One day after tumor cell inoculation, the mice were given a bolus intravenous injection of indicated drug via the lateral tail vein. Control groups were injected with saline. Free drug samples were prepared on the day of injection such that the final concentrations were sufficient to deliver the indicated drug dose in a volume of 200 μl. All dilutions were made using 0.9% Sodium Chloride Injection USP. The mice were briefly (less than 30 sec.) restrained during intravenous injections. Dilation of the vein was achieved by holding the animals under a heat lamp for a period of between five and ten minutes. Following administration of the test articles, animals were weighed daily for fourteen days and monitored for signs of stress twice daily for the first 14 days (once daily on weekends) and once daily for the remainder of the study. Severely distressed animals were terminated by CO₂ asphyxiation and the time of death was recorded to occur on the following day. Although complete dose titrations were completed for each drug, the data shown in FIG. 6 is that obtained after administration of the free drugs at their maximum tolerated dose. This was 3, 40 and 40 mg/kg for vincristine, Navelbine™ and AHVB, respectively.

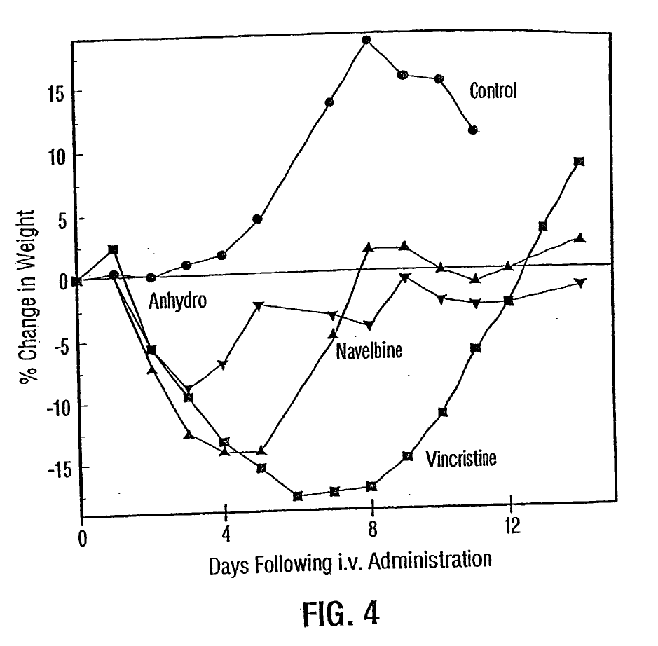

FIG. 4 presents the results of a study demonstrating vinca alkaloid induced weight loss following a single intravenous injection of the indicated drug at the maximum tolerated dose (see FIG. 6). These data were obtained as part of the study detailed in FIG. 6. After treating mice (bearing the P388 tumor) with a single dose of the indicated drug, animals were examined twice daily for the first 14 days (once daily on weekends). Mean body weight was determined daily over this time period and the results are shown in FIG. 4. Weight gain in the control is an indication of tumor progression. Results indicate that AHVB, administered at 40 mg/kg, is the least toxic of the three drugs evaluated.

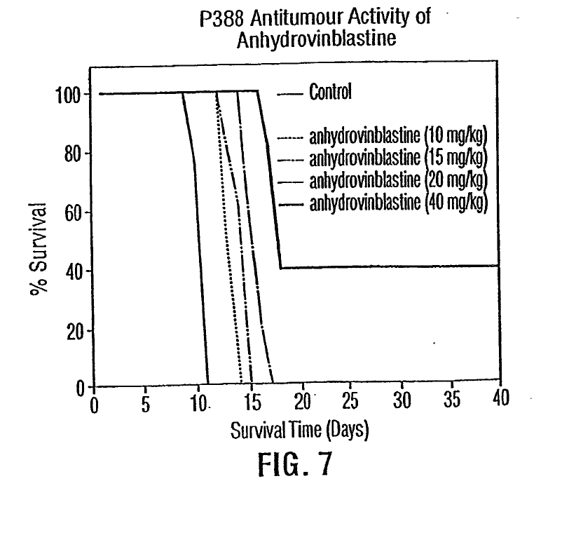

The dose response curve obtained for AHVB when used to treat BDF1 mice bearing P388 tumors is presented in FIG. 7. The studies were conducted as described for FIG. 6. The maximum tolerated dose of AHVB (40 mg/kg) as specified in these studies reflects a very acute (within 1 hour) toxic reaction that limits further dose excalatin for IV administration of AHVB. This contrasts the more prolonged toxicity observed for Navelbine™ at its maximum tolerated dose and suggests that an ability to circumvent the acute toxicity of AHVB could lead to significant increases in its maximum tolerated dose.

Based on observation of the in vitro drug screen studies, it is surprising that AHVB would perform well as an antineoplastic agent for use in cancer therapy. The in vitro tests indicate that AHVB is consistently 10 to 15 fold less active on per molar basis (Table I and FIG. 5) than vincristine and Navelbine™. These results suggest that AHVB would not perform well as an anti-tumor agent. However, in an efficacy study, also employing the P388 cell line (see FIG. 6), the anti-tumor activity of AHVB at the maximum tolerated dose (40 mg/kg, single IV injection) is significantly better than that observed for vincristine (administered at the maximum tolerated dose of the free drug of 3 mg/kg). Improved anti-tumor activity, in this case, is measured by the number of long term survivors (>60 days). It is important to stress that, for this example, AHVB is approximately 10 times less toxic (on a weight basis) than vincristine. Therefore, 10 times more drug can be given and it is at this dose that improvements were observed in the long term survival of animals with P388 tumors. When compared to Navelbine™, the in vivo results are even more surprising as the maximum tolerated dose of the two drugs in animals bearing P388 tumors are about the same (40 mg/kg).

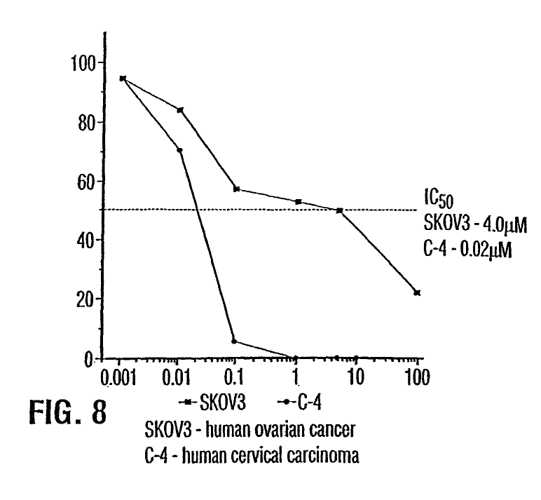

FIG. 8 shows the cytotoxicity of AVHB on SK0V3 cells and C-4 cells with a 3 day incubation. The IC₅₀s for the SK0V3 and C-4 cells were 4.0 μM and 0.02 μM respectively. Both cell lines were obtained from the ATCC and grown using standard growth techniques and medium as described above. The IC₅₀s were determined through standard cytoxicity assays described above, with each well containing approximately 10⁴ cells.

Studies in the H460 SC Tumor Mouse Model

Cultures of H460 Human Lung cells are available from the British Columbia Cancer Research Center. Cells were injected subcutaneously twice into mature male Rag-2 mice (24 mice, 1×10⁶ cells/mouse) using a 26-gauge needle. The H460 cells were suspended in a Hank's Balanced Salt Solution without calcium. Tumors were allowed to form in the mice for 11 days.

When the tumors were well established, four separate groups of mice, were selected for administration of the three test articles (i.e. one group for each test article of AHVB bisulphate, AHVB ditartrate, and Navelbine™) and one control.

AHVB bisulphate and ditartrate, and Navelbine™ were solubilized using 5% dextrose saturated with Argon. Both of these articles were at a concentration of 20 mg/ml. Any dose dilutions were made with 5% dextrose.

The articles were administered intravenously on the days 1, 5 and 9, as were controls of 5% dextrose. Body weights and tumor measurements with calipers were taken every day for the first 10 days and then every other day for the remainder of the study.

Following administration of the test articles, the animals; weight and tumor size (using calipers) were determined daily for the first 10 days and then every other day for the remainder of the study. If the tumor size reached 1 gram in weight or the tumor started to ulcerate, the animals were sacrificed (by carbon dioxide inhalation) and subjected to necropsy. Animals were also monitored at least daily for signs of stress for the full length of the study. Animals manifesting severe symptoms of stress (rapid weight loss, panting, hunched posture, scruffy coat) were also sacrificed and a necropsy performed.

Anhydrovinblastine Sulfate (3′,4′-dehydrovinblastine) was obtained from the British Columbia Cancer Agency (BCCA), Investigational Drug Section. Navelbine™ (vinorelbine tartrate; 3′,4′-didehydro-4′-deoxy-C′-norvincaleukoblastine-di-L-tartrate) was purchased from Glaxo/Burroughs Wellcome Inc., Canada.

The methodology involving animals was approved by the BCCA's Institutional Animal Care Committee (IACC) at UBC prior to conducting the studies (Animal Care Certificate No. A94-1602). During the study the care, housing and use of animals was performed in accordance with the Canadian Council on Animal Care Guidelines.

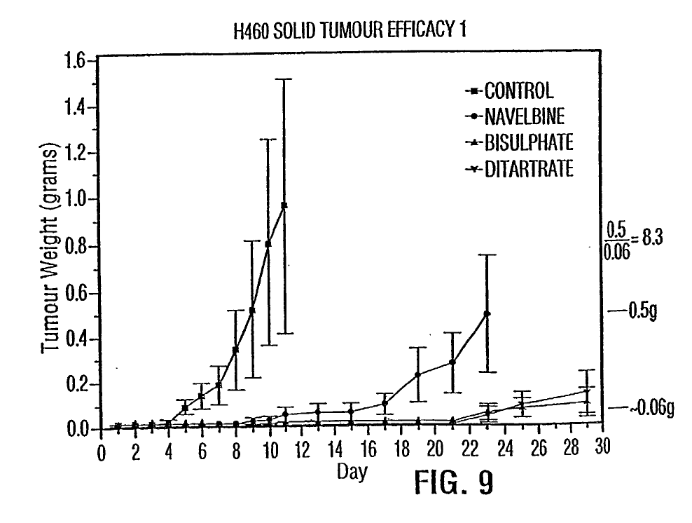

The results of the efficacy studies are given in FIG. 9 and present averages of data from 6 or fewer animals. Each mouse in a given article group had two subcutaneous tumors on its back. Each tumor was measured in length and width and the volume of each tumor was calculated by (L×W)²/2. The two tumor volumes were then averaged. The volume averages of all the mice/group were averaged to yield a mean for the single date point appears on the graph in FIG. 9. The calculation was performed each day the tumors were measured. The standard deviation of the mean and the standard error of the mean were calculated with the error bars appearing in the graph in FIG. 9.

Studies in the C-4 (Cervical) Solid Tumor Model

Cultures of C-4 Human Cervical Carcinoma cells are available from the British Columbia Cancer Research Centre. Cells were injected subcutaneously twice into mature male Rag-2 mice (24 mice, 1×10⁶ cells/mouse) using a 26-gauge needle. The C-4 cells were suspended in a Hank's Balanced Salt Solution without calcium. Tumors were allowed to form in the mice for 31 days.

When the tumors were well established, four separate groups of mice, were selected for administration of the three test articles (i.e. one group for each test article of AHVB bisulphate, AHVB ditartrate, and Navelbine™) and one control.

AHVB bisulphate and ditartrate, and Navelbine™ were solubilized using 5% dextrose saturated with Argon. These articles were administered at doses of 20 mg/Kg intravenously. Any dose dilutions were made with 5% dextrose.

The articles were administered intravenously on the days 1, 5 and 9, as were controls of 5% dextrose. Body weights and tumor measurements with calipers were taken regularly over the period of the study of 69 days.

Following administration of the test articles, the animals; weight and tumor size (using calipers) were determined regularly over the period of the study. If the tumor size reached I gram in weight or the tumor started to ulcerate, the animals were sacrificed (by carbon dioxide inhalation) and subjected to necropsy. Animals were also monitored at least daily for signs of stress for the full length of the study. Animals manifesting severe symptoms of stress (rapid weight loss, panting, hunched posture, scruffy coat) were also sacrificed and a necropsy performed.

Anhydrovinblastine Sulfate (3′,4′-dehydrovinblastine) was obtained from the British Columbia Cancer Agency (BCCA), Investigational Drug Section. Navelbine™ (vinorelbine tartrate; 3′,4′-didehydro-4′-deoxy-C′-norvincaleukoblastine-di-L-tartrate) was purchased from Glaxo/Burroughs Wellcome Inc., Canada.

The methodology involving animals was approved by the BCCA's Institutional Animal Care Committee (IACC) at UBC prior to conducting the studies (Animal Care Certificate No. A94-1602). During the study the care, housing and use of animals was performed in accordance with the Canadian Council on Animal Care Guidelines.

The results of the efficacy studies are given in Table 3 and present averages of data from 6 or fewer animals. Each mouse in a given article group had two subcutaneous tumors on its back. Each tumor was measured in length and width and the volume of each tumor was calculated by (L×W)²/2. The two tumor volumes were then averaged. The volume averages of all the mice/group were averaged to yield a mean for each single date point. The calculation was performed each day the tumors were measured.

Navelbine™ tumors reached their observable ‘growth threshold’ at day 41 and continued to grow steadily whereas the AHVB ditartrate reached the threshold on day 55. The tumor treated with AHVB bisulphate showed negligible tumor growth through day 69. NavelbineT^{m} had an 84% delay in growth in the tumor, AHVB ditartrate had an extended delay of 106%, and AHVB bisulphate exhibited a marked delay in tumor growth of greater than 209%. Tumor growth did not reach the observable growth threshold over 70 days. This data is found in Table 3.

Taken together, the results presented here show that AHVB has significant and unique pharmacological properties in vivo that lead to significant improvements in in vivo antitumor efficacy relative to other vinca alkaloids such as vincristine and Navelbine™. These results are unique and new in that the in vivo activity of AHVB predicted it to be significantly less on the basis of in vitro cytotoxicity studies.

Clinical Trials in Cancer Patients

One skilled in the art will appreciate that, following the demonstrated effectiveness of AHVB in vitro and in animal models, AHVB should be tested in Clinical Trials in order to further evaluate its efficacy in the treatment of cancer and to obtain regulatory approval for therapeutic use. As is known in the art, clinical trials progress through phases of testing, which are identified as Phases I, II, III, and IV.

Initially AHVB will be evaluated in a Phase I trial. Typically Phase I trials are used to determine the best mode of administration (for example, by pill or by injection), the frequency of administration, and the toxicity for the compounds. Phase I studies frequently include laboratory tests, such as blood tests and biopsies, to evaluate the effects of a compound in the body of the patient. For a Phase I trial, a small group of cancer patients are treated with a specific dose of AHVB. During the trial, the dose is typically increased group by group in order to determine the maximum tolerated dose (MTD) and the dose-limiting toxicities (DLT) associated with the compound. This process determines an appropriate dose to use in a subsequent Phase II trial.

A Phase II trial can be conducted to further evaluate the effectiveness and safety of AHVB. In Phase II trials, AHVB is administered to groups of patients with either one specific type of cancer or with related cancers, using the dosage found to be effective in Phase I trials.

Phase III trials focus on determining how a compound compares to the standard, or most widely accepted, treatment. In Phase III trials, patients are randomly assigned to one of two or more “arms”. In a trial with two arms, for example, one arm will receive the standard treatment (control group) and the other arm will receive AHVB treatment (investigational group).

Phase IV trials are used to further evaluate the long-term safety and effectiveness of a compound. Phase IV trials are less common than Phase I, II and III trials and will take place after AHVB has been approved for standard use.

Eligibility of Patients for Clinical Trials

Participant eligibility criteria can range from general (for example, age, sex, type of cancer) to specific (for example, type and number of prior treatments, tumor characteristics, blood cell counts, organ function). Eligibility criteria may also vary with trial phase. For example, in Phase I and II trials, the criteria often exclude patients who may be at risk from the investigational treatment because of abnormal organ function or other factors. In Phase II and III trials additional criteria are often included regarding disease type and stage, and number and type of prior treatments.

Phase I cancer trials usually comprise 15 to 30 participants for whom other treatment options have not been effective. Phase II trials typically comprise up to 100 participants who have already received chemotherapy, surgery, or radiation treatment, but for whom the treatment has not been effective. Participation in Phase II trials is often restricted based on the previous treatment received. Phase III trials usually comprise hundreds to thousands of participants. This large number of participants is necessary in order to determine whether there are true differences between the effectiveness of AHVB and the standard treatment. Phase III may comprise patients ranging from those newly diagnosed with cancer to those with extensive disease in order to cover the disease continuum.

One skilled in the art will appreciate that clinical trials should be designed to be as inclusive as possible without making the study population too diverse to determine whether the treatment might be as effective on a more narrowly defined population. The more diverse the population included in the trial, the more applicable the results could be to the general population, particularly in Phase III trials. Selection of appropriate participants in each phase of clinical trial is considered to be within the ordinary skills of a worker in the art.

Assessment of Patients Prior to Treatment

Prior to commencement of the study, several measures known in the art can be used to first classify the patients. Patients can first be assessed, for example, using the Eastern Cooperative Oncology Group (ECOG) Performance Status (PS) scale. ECOG PS is a widely accepted standard for the assessment of the progression of a patient's disease as measured by functional impairment in the patient, with ECOG PS 0 indicating no functional impairment, ECOG PS 1 and 2 indicating that the patients have progressively greater functional impairment but are still ambulatory and ECOG PS 3 and 4 indicating progressive disablement and lack of mobility.

Patients' overall quality of life can be assessed, for example, using the McGill Quality of Life Questionnaire (MQOL) (Cohen et al (1995) Palliative Medicine 9: 207-219). The MQOL measures physical symptoms; physical, psychological and existential well-being; support; and overall quality of life. To assess symptoms such as nausea, mood, appetite, insomnia, mobility and fatigue the Symptom Distress Scale (SDS) developed by McCorkle and Young ((1978) Cancer Nursing 1: 373-378) can be used.

Patients can also be classified according to the type and/or stage of their disease and/or by tumor size.

Administration of AHVB in Clinical Trials

AHVB is typically administered to the trial participants parenterally. In one embodiment, AHVB is administered by intravenous infusion. Methods of administering drugs by intravenous infusion are known in the art. Usually intravenous infusion takes place over a certain time period, for example, over the course of 60 minutes.

A range of doses of AHVB can be tested. The studies outlined above have indicated that AHVB can be safely administered at concentrations up to ten times the dosages typically used for vinblastine. Provided with this information and effective dosages of vinblastine known in the art, a skilled practitioner could readily determine appropriate dosages of AHVB for use in clinical trials. An exemplary dose range for AHVB treatment includes dosages in the range 2.5 mg/M² to 30 mg/M².

Pharmacokinetic Monitoring

To fulfil Phase I criteria, distribution of the AHVB is monitored, for example, by chemical analysis of samples, such as blood or urine, collected at regular intervals. For example, samples can be taken at regular intervals up until about 72 hours after the start of infusion. In one embodiment, samples are taken at 0, 0.33, 0.67, 1, 1.25, 1.5, 2, 4, 6, 8, 12, 24, 48 and 72 hours after the start of each infusion of AHVB.

If analysis is not conducted immediately, the samples can be placed on dry ice after collection and subsequently transported to a freezer to be stored at −70° C. until analysis can be conducted. Samples can be prepared for analysis using standard techniques known in the art and the amount of AHVB present can be determined, for example, by high-performance liquid chromatography (HPLC).

Pharmacokinetic data can be generated and analyzed in collaboration with an expert clinical pharmacologist and used to determine, for example, clearance, half-life and maximum plasma concentration.

Monitoring of Patient Outcome

The endpoint of a clinical trial is a measurable outcome that indicates the effectiveness of a compound under evaluation. The endpoint is established prior to the commencement of the trial and will vary depending on the type and phase of the clinical trial. Examples of endpoints include, for example, tumor response rate—the proportion of trial participants whose tumor was reduced in size by a specific amount, usually described as a percentage; disease-free survival—the amount of time a participant survives without cancer occurring or recurring, usually measured in months; overall survival—the amount of time a participant lives, typically measured from the beginning of the clinical trial until the time of death. For advanced and/or metastatic cancers, disease stabilization—the proportion of trial participants whose disease has stabilized, for example, whose tumor(s) has ceased to grow and/or metastasize, can be used as an endpoint. Other endpoints include toxicity and quality of life.

Tumor response rate is a typical endpoint in Phase II trials. However, even if a treatment reduces the size of a participant's tumor and lengthens the period of disease-free survival, it may not lengthen overall survival. In such a case, side effects and failure to extend overall survival might outweigh the benefit of longer disease-free survival. Alternatively, the participant's improved quality of life during the tumor-free interval might outweigh other factors. Thus, because tumor response rates are often temporary and may not translate into long-term survival benefits for the participant, response rate is a reasonable measure of a treatment's effectiveness in a Phase II trial, whereas participant survival and quality of life are typically used as endpoints in a Phase III trial.

Phase I Clinical Trials

In preclinical studies involving human tumor xenografts of non-small cell lung cancer (NSCLC) and cervical cancer, AHVB showed superior activity to that of both vincristine and vinorelbine at equitoxic doses. Toxicological studies in rats and dogs demonstrated reversible myelosupression and gastrointestinal toxicities. Based on these data, a Phase I trial was undertaken to determine the feasibility of administering AHVB as a 1 hr intravenous (IV) infusion once every 3 weeks to patients with advanced refractory solid tumors, in order to determine the maximum-tolerated dose (MTD), the dose limiting toxicity (DLT) and to evaluate the major pharmacokinetic parameters.

Patients had normal bone marrow, hepatic and renal function. Twenty-four patients were treated with escalating doses of AHVB, administered as a 1 hour infusion every 3 weeks. The 24 patients comprised 12 male and 12 female patients with a median age of 60 years (range 27-75 years). Twenty-one of the 24 patients were evaluable. Diagnoses were non-small cell lung cancer (NSCLC), colorectal cancer, soft tissue sarcoma, pancreatic cancer, breast cancer and metastatic neuroendocrine cancer in 11, 5, 4, 1, 1, and 1 patient(s), respectively. Patients have had a median of 3 chemotherapy regimens (range 1-6). A total of 51 courses were administered at doses of 2.5, 5, 10,16.5, 21,25 and 30 mg/m² to 1,3, 1,3, 6, 6 and 1 patient(s), respectively (see Table 4).

The first patient was entered in the trial at the 2.5 mg/m² dose level. At the next dose level (5 mg/m²), three patients were enrolled because one patient developed non-dose limiting toxicities in the form of Grade 2 anorexia, hyperamylasemia, and increased serum creatinine. Five patients were enrolled at the 16.5 mg/m² dose level because two patients were not evaluable and had to be replaced. Grade 2 toxicities including infusional hypertension, anemia and dizziness were noted at the 16.5 mg/m² dose level. Six patients were enrolled at the 25 mg/m² dose level. DLTs including Grade 4 level constipation and Grade 3 nausea and vomiting were noted at this dose level. This dose level, therefore, exceeded the MTD.

Since minimal toxicities were seen in patients enrolled at the 16.5 mg/m² dose level and the increment from 16.5 to 25 mg represented a 50% increase in dose, it was elected to evaluate an intermediate dose level of 21 mg/m². Seven patients were enrolled at the 21 mg/m² dose level, but only 6 were evaluable for toxicity due to one patient developing Grade 2 hypertension, headache, nausea and vomiting at the start of the infusion, which recurred on rechallenge. This patient received only 15 ml of the drug solution and, therefore, was not evaluable to assess toxicity. One patient at this dose level had Grade 3 nausea and vomiting and Grade 2 constipation, requiring brief hospitalisation, laxatives and administration of intravenous fluids. The nausea and vomiting observed at the 21 mg/m² dose level was a DLT. It was determined that the 21 mg/m² dose is the MTD.

Stable disease was noted in one patient with metastatic sarcoma to lungs at a dose level of 10 mg/M². Stable disease was also noted in three patients with metastatic NSCLC at dose levels of 21 and 25 mg/m².

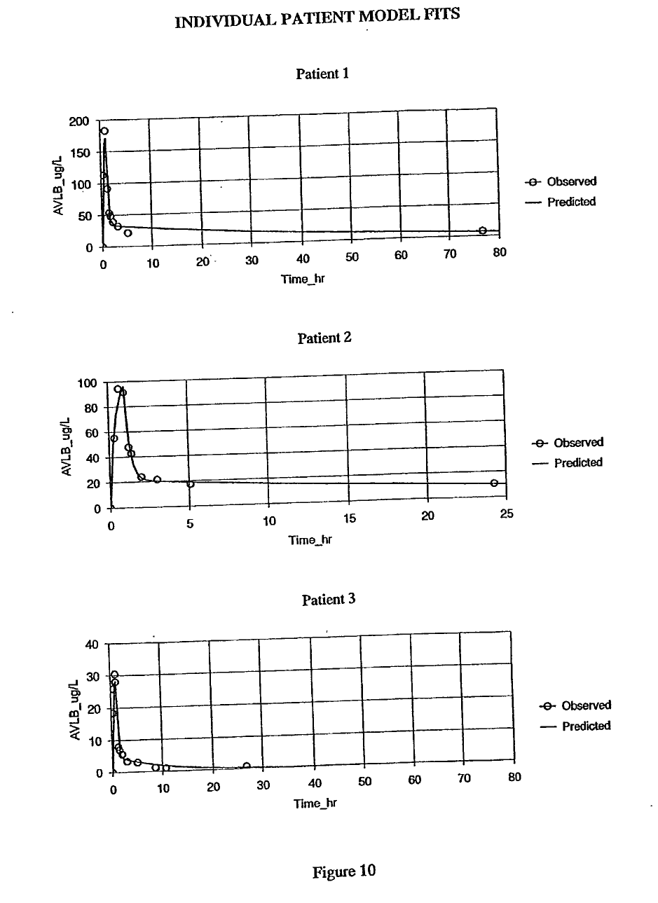

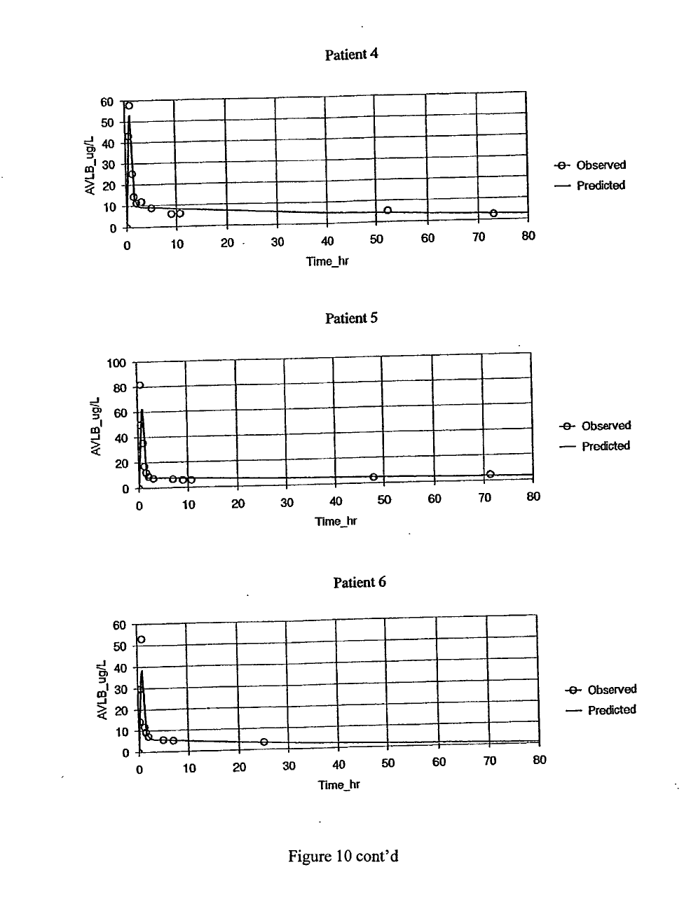

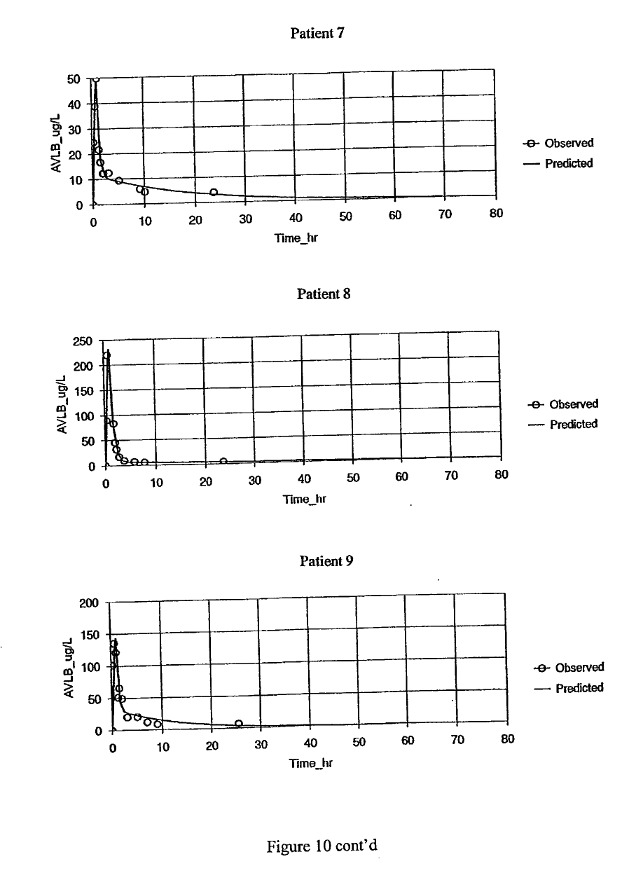

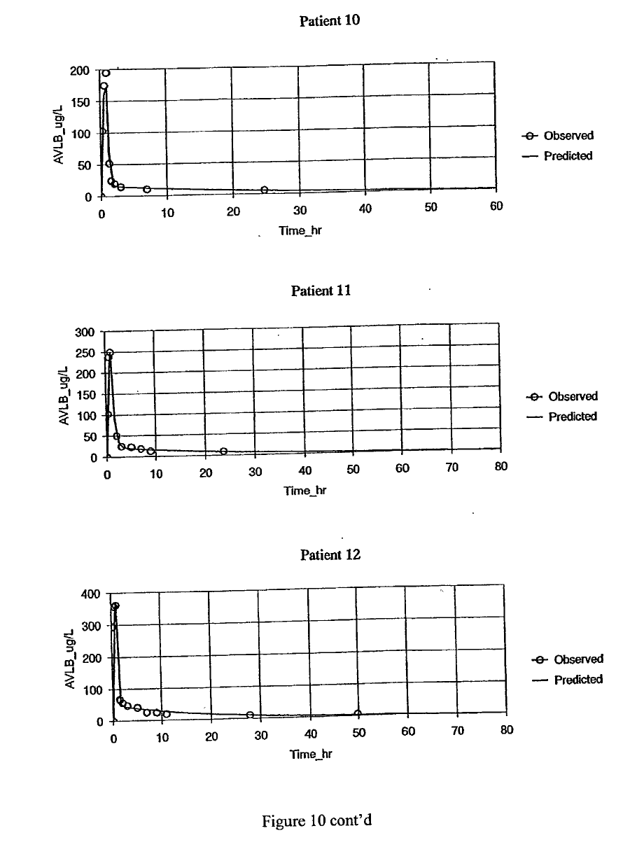

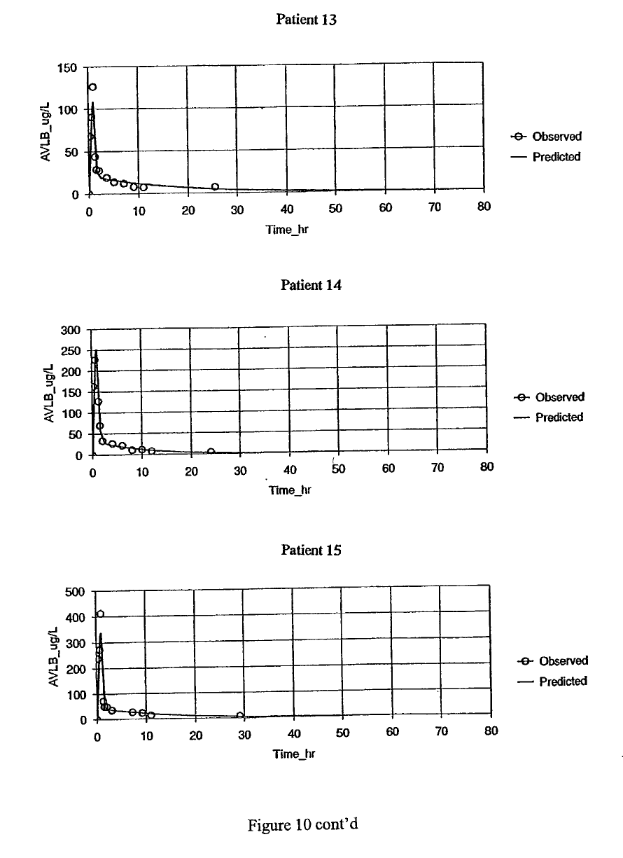

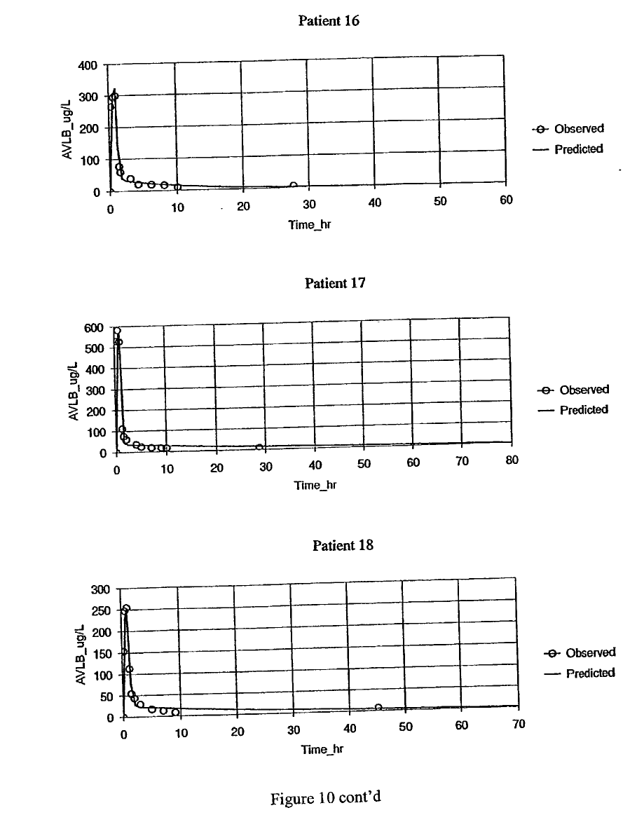

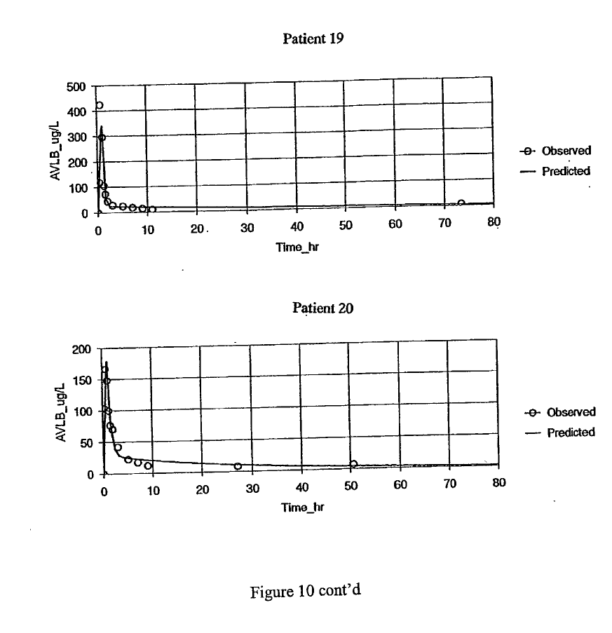

AHVB blood serum concentrations were measured at 0, 0.33, 0.67, 1, 1.25, 1.5, 2, 4, 6, 8, 12, 24, 48, and 72 hrs after the start of each infusion. FIG. 10 shows the time-course plots of individual patient AHVB serum concentrations. The serum extracts were assayed using high pressure liquid chromatography (HPLC) and were fit to a 2-compartment pharmacokinetic model for determination of pharmacokinetic parameters (see Table 5).

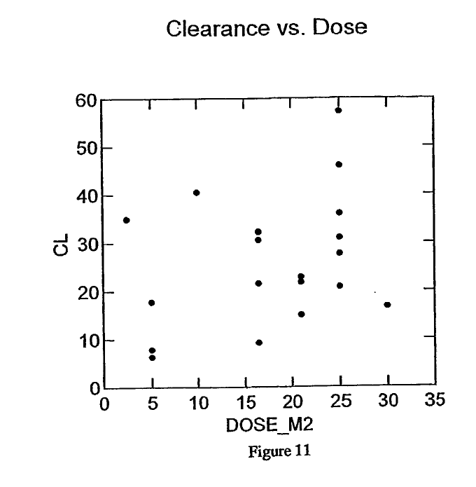

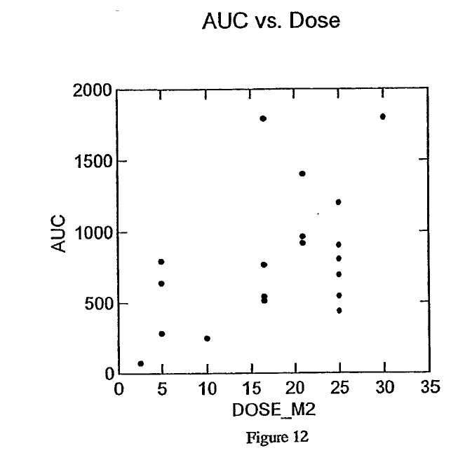

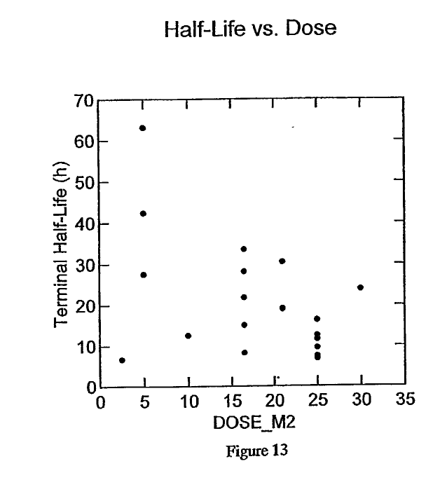

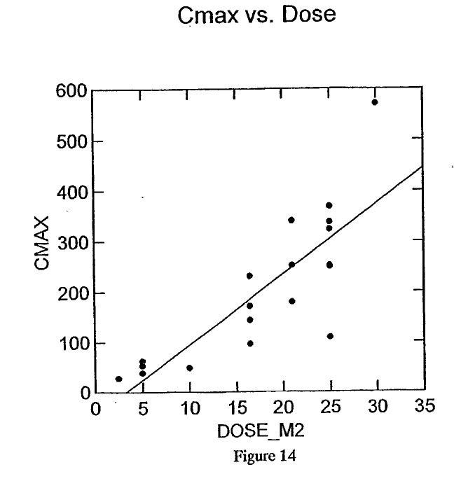

The pharmacokinetics of AHVB are linear, and well characterized by a 2-compartment model, with mean values for clearance of 26.4 L/h/m², alpha half-life of 0.19 h, beta half-life of 20.8 h and Vₛₛ of 451 L/m². There appears to be no significant change in clearance observed between smaller and larger doses (p>0.2 by linear regression). FIG. 11 shows the clearance of AHVB for each patient at their respective dose level. Similarly, AUC appeared to increase linearly with dose (r²=0.82, p<0.05) (FIG. 12). A similar finding was observed with the maximum plasma concentration (Cₘₐₓ) (FIG. 13). The variability in clearance and volume of distribution was modest, with a CV % of 49% and 39%, respectively.

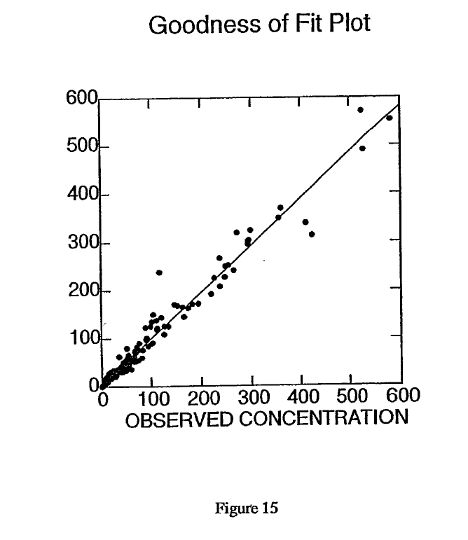

A goodness of fit plot is shown in FIG. 15 demonstrating the model predicted versus measured AHVB concentrations. A minority of patients showed evidence to support a third elimination phase, however, this could not be completely characterised in this study. Vinblastine has been reported to have a triphasic elimination profile, with the initial alpha half-life of less than 5 minutes. The current study precluded an evaluation of this rapid phase, if it exists for AHVB. Otherwise the pharmacokinetics of AHVB were similar to that reported for vinblastine.

Pharmaceutical Preparations

The present invention also provides pharmaceutical compositions containing AHVB in combination with one or more pharmaceutically acceptable, inert or physiologically active, carriers, diluents or adjuvants. AHVB can be freeze dried and, if desired, combined with other pharmaceutically acceptable excipients to prepare formulations for administration. If desired, the pharmaceutical compositions comprising AHVB may further comprise one or more other active ingredients, for example, other chemotherapeutic agents useful in the treatment of cancer. These compositions may be presented in any form appropriate for the administration route envisaged. In one embodiment of the invention, AHVB is formulated for parenteral administration. In another embodiment, AHVB is formulated for intravenous administration.

AHVB may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrastemal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose, hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propylp-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 1,3-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables. When AHVB is administered parenterally in a sterile medium or vehicle, it can either be suspended or dissolved in the vehicle depending on the vehicle and concentration used. Advantageously, adjuvants such as local anaesthetics, preservatives and buffering agents can also be dissolved in the vehicle.

AHVB may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philadelphia, Pa. (2000) (formerly “Remingtons Pharmaceutical Sciences”).

Administration of AHVB

AHVB may be administered in a number of ways depending upon whether local or systemic treatment of the organism is desired. Administration may be pulmonary, e.g. by inhalation or insufflation of powders or aerosols, including by nebulizer; intratracheal; intranasal; epidermal or transdermal; oral or parenteral. Parenteral administration includes intravenous, intraarterial, subcutaneous, intraperitoneal or intramuscular injection or infusion, or intracranial, e.g. intrathecal or intraventricular, administration. For parenteral injection, AHVB or pharmaceutical compositions comprising AHVB are used in the form of a sterile solution containing other solutes, for example, enough saline or glucose to make the solution isotonic. In one embodiment of the present invention, AHVB is administered by parenteral infusion. In another embodiment, AHVB is administered through intravenous (IV) infusion.

AHVB may be administered topically in a lotion or cream, for example, for application to the skin in order to treat a melanoma.

For administration to an individual for the treatment cancer, the present invention also contemplates the formulation of AHVB into oral dosage forms such as tablets, capsules and the like as described above. In all cases, the proportion of active ingredients in any solid and liquid composition will be at least sufficient to impart the desired activity to the individual being treated upon oral administration.

For administration by inhalation or insufflation, AHVB or pharmaceutical compositions comprising AHVB can be formulated into an aqueous or partially aqueous solution, which can then be utilized in the form of an aerosol.

The dose of AHVB to be administered, whether a single dose, multiple dose, or a daily dose, will vary and a dose regimen is designed based on such factors as the potency of the compound, the particular compositions employed, route of administration, size of the patient and the nature and severity of the patient's condition, amongst others. The dosage to be administered is not subject to defined limits, but it will usually be an effective amount. It will also usually be the equivalent, on a molar basis, of the pharmacologically active free form produced from a dosage formulation upon the metabolic release of the active free drug to achieve its desired pharmacological and physiological effects.

Dosage requirements can be determined by standard clinical techniques, known to a worker skilled in the art. Treatment will generally be initiated with small dosages less than the optimum dose of the compound. Thereafter the dosage is increased until the optimum effect under the circumstances is reached. In general, AHVB or pharmaceutical compositions comprising AHVB is administered at a concentration that will generally afford effective results while minimizing harmful or deleterious side effects. Administration can be either as a single unit dose or, if desired, the dosage can be divided into convenient sub-units that are administered at suitable times throughout the day. An oncologist skilled in the art of cancer treatment will be able to ascertain, without undue experimentation, appropriate protocols for effective administration of the compounds of this present invention by referring to the earlier studies of vinblastine and its derivatives.

In one embodiment of the present invention, AHVB is administered at a dose between about 2 mg/m² and about 30 mg/m². In another embodiment, AHVB is administered at a dose between about 2.5 mg/m² and about 25 mg/m². In another embodiment, AHVB is administered at a dose between about 5 mg/m² and about 25 mg/m². In another embodiment, AHVB is administered at a dose between about 10 mg/m² and about 25 mg/m². In another embodiment, AHVB is administered at a dose between about 10 mg/m² and about 21 mg/m². In another embodiment, AHVB is administered at a dose between about 16.5 mg/m² and about 21 mg/m².

When AHVB is administered by intravenous infusion, the dose of AHVB is typically administered to the patient over a time period of between about 30 minutes and about 90 minutes. In one embodiment of the present invention, AHVB is administered by intravenous infusion over a time period of about 60 minutes.

Treatment regimens are typically designed such that the AHVB is administered to the patient in cycles. Treatment with AHVB in accordance with the present invention may be part of a treatment regimen that involves one cycle of administration or the regimen may involve more than one cycle. Generally, the treatment regimen involves between about 2 and about 10 cycles. In one embodiment of the present invention, the treatment regimen involves between about 4 and about 8 cycles. In another embodiment, the treatment regimen involves about 6 cycles. Typically, a cycle is between about 1 and about 4 weeks. In one embodiment, the cycle is about 3 weeks.

Therapeutic Uses

AHVB, a derivative of the vinca alkaloid vinblastine, has shown significant cytotoxic potential against a panel of human cancer cell lines, and significant activity against the human H460 non-small cell lung carcinoma tumor xenograph in SCID/Rag-2 Mice. In vitro cytotoxicity assays utilizing the MTT cytotoxicity assay with a drug exposure time of 72 hours have shown that AHVB is an active cytotoxic drug with IC₅₀ values ranging from 20-24 nM against the H460 human non-small cell lung carcinoma, C-4 human cervical carcinoma, K562 human leukaemia, and the A431 human epidermoid cell lines. AHVB was approximately 10-fold less active than Navelbine™ when tested in vitro against the same cell lines. Surprisingly, however when AHVB was tested in vitro in solid tumor efficacy experiments, it was found to be more potent than Navelbine™. Male SCID/Rag-2 mice were inoculated subcutaneous with H460 cells and after 12 days of tumor growth AHVB and Navelbine™ were delivered IV at doses of 10 mg/kg and 20 mg/kg on days 1, 5 and 9. In this model, AHVB resulted in greater tumor growth inhibition and was less toxic than Navelbine™. These results indicate that AHVB has desirable pharmacological properties for therapeutic applications.

In clinical trials, AHVB was shown to be active in stabilising cancer in humans. Phase I clinical trials in which AHVB was administered to patients as a 60 minute intravenous infusion once every 3 weeks for up to 6 courses at a dose of 10 mg/m² demonstrated stable disease in one patient with metastatic sarcoma to the lungs. At dosage levels of 21 and 25 mg/m², stable disease was shown in three patients with metastatic NSCLC. These results demonstrate that AHVB has a significant effect in humans in the treatment of cancer.

In accordance with the present invention, AHVB may be used as part of a neo-adjuvant therapy (to primary therapy), as part of an adjuvant therapy regimen, and/or for the treatment of locally advanced or metastatic disease. The term “metastatic disease” refers a disease that has spread from one part of the body to another. AHVB may also be used to treat refractory and/or advanced tumors. It is further contemplated that AHVB can be used to treat patients that have undergone one or more prior courses of chemotherapy.

Primary therapy is understood to encompass a first line of treatment upon the initial diagnosis of cancer in a patient. Exemplary primary therapies may involve surgery, a wide range of chemotherapies and radiotherapy.

Adjuvant therapy is understood to encompass any therapy, following a primary therapy such as surgery that is administered to patients at risk of relapsing. Adjuvant systemic therapy is begun soon after primary therapy to delay recurrence, prolong survival or cure a patient. It is contemplated that AHVB can be used alone or in combination with one or more other chemotherapeutic agents as part of an adjuvant therapy.

In the application of cancer therapies a patient's response status is monitored to determine the effectiveness of the therapy. “Response status” refers to measurement of changes in the tumor(s) or lesion(s) under chemotherapy, namely any observed growth (progression of disease), stability, or shrinkage (complete or partial response). Arising out such monitoring may be the observation of relapse in a patient, which may refer to the relapse of a patient with advanced disease. “Relapse time” is the time from the initial appearance of a primary cancer to the appearance of advanced disease requiring chemotherapy.

“Advanced disease,” as used herein, refers to overt disease in a patient, wherein such overt disease is not amenable to cure by local modalities of treatment, such as surgery or radiotherapy. Advanced disease may refer to a locally advanced cancer or it may refer to metastatic disease. In one embodiment of the present invention, AHVB is used alone or in combination with one or more other chemotherapeutics in the treatment of advanced disease in a cancer patient. In another embodiment, the advanced disease is a solid tumor. In another embodiment, the advanced disease is a metastatic disease. In another embodiment, the advanced disease is metastatic sarcoma. In other embodiments, the advanced disease is metastatic neuroendocrine cancer or metastatic non-small cell lung carcinoma.

The progression of advanced disease is monitored to help evaluate when chemotherapy may be appropriate and may be marked by an increase of at least 25% in the overall sum of measurable lesions as compared to nadir (i.e. best response) and/or the appearance of new lesions following primary therapy. Alternatively, lesions may be found to shrink in size.

A “refractory” cancer or tumor refers to a cancer or tumor that has not responded to treatment. In accordance with the present invention, AHVB can be used to treat a refractory cancer. In one embodiment, AHVB is used to treat refractory non-small cell lung carcinoma. In another embodiment, AHVB is used to treat refractory colorectal carcinoma. In another embodiment, AHVB is used to treat refractory soft tissue sarcoma. In another embodiment, AHVB is used to treat refractory pancreatic cancer. In another embodiment, AHVB is used to treat refractory breast cancer.

It is to be understood that the examples described above are not meant to limit the scope of the present invention. It is expected that numerous variants will be obvious to the person skilled in the art to which the present invention pertains, without any departure from the spirit of the present invention. The appended claims, properly construed, form the only limitation upon the scope of the present invention.

TABLE 1
Relative Cytotoxicity of Vincristine, AHVB and Navelbine ™ on Tumor
Cell Lines

| | | Ex- | | | |
|---|---|---|---|---|---|
| | | posure | | | |
| Cell | | Time | Drug Ic₅₀ (nM) | | |
| Line | Type | (Days) | Vincristine | Navelbine | AHVB |
| P388 | murine | 1 | 11.0 ± 3.6 | 20.0 ± ∩0.0 | 140.0 ± 53.0 |
| | leukaemia | 3 | 1.0 ± 0.3 | 0.7 ± 0.3 | 15.0 ± 8.7 |
| | | 7 | 2 | 2.5 | 20 |
| MCF7 | human | 1 | N.D. | N.D. | N.D. |
| | breast | 3 | >2500 | >2500 | >2500 |
| | | 7 | 2.6 ± 1.6 | 2.6 ± 1.6 | 31.3 ± 12.4 |
| H460 | human | 1 | N.D. | N.D. | N.D. |
| | lung | 3 | 3.5 | 0.3 | 10 |
| | | 7 | 2.5 | >0.5 | 5 |
| K562 | human | 1 | >50.0 | >50.0 | >50.0 |
| | erythro- | 3 | 1.5 ± 0.4 | 2.5 ± 2.2 | 18.8 ± 8.8 |
| | leukaemia | 7 | N.D. | N.D. | N.D. |
| LS180 | human | 1 | N.D. | N.D. | N.D. |
| | colon | 3 | >50.0 | >50.0 | >50.0 |
| | | 7 | 1.5 | 0.5 | 17.5 |

TABLE 2
Estimation of Subacutely Toxic Dosages of Vincristine Sulfate,
Navelbine ™, and AHVB when administered to Healthy Male Nb
Rats as a Single Intraperitoneal Injection

| | | Mortality (surviving rats/injected |
|---|---|---|
| Drug | Dose (mg/kg) | rats) |
| 1 ml Saline pH 4.3 | n/a | 35856 |
| Vineristine sulfate | 1 | 0 |
| | 0.7 | 35856 |
| | 0.6 | 35856 |
| | 0.5 | 35856 |
| Navelbine ™ | 10 | 0 |
| (Vinorelbine tartrate) | 5 | 0 |
| | 3 | 35828 |
| | 2 | 35856 |
| | 1 | 35856 |
| Anhydrovinblastine | 10 | 0 |
| | 5 | 35828 |
| | 4.4 | 0 |
| | 4 | 35796 |
| | 3 | 35856 |

TABLE 3
Solid Tumor Delay in Growth Data

| | | Initial Growth | | |
|---|---|---|---|---|
| | | (Day) | | % Delay In |
| Experiment | Dose¹ | Total | Of Expt. | Growth (Dig) |
| C-4eff1 | Control (Saline) | 32 | 2 | |
| | Navelbine ™ | 59 | 29 | 84 |
| | AHVB Bisulphate | 99 | 69 | 209 |
| | AHVB Ditartrate | 66 | 36 | 106 |

¹dose = 20 mg/kg intravenously on days 1, 5, 9

TABLE 4
Patient dosage levels and associated toxicities

| | Dose | |
|---|---|---|
| Dose | Concentration | |
| Level | (mg/m²) | Patient(s) treated |
| 1 | 2.5 | One patient treated |
| 2 | 5.0 | One patient treated developed Grade 2 elevated amylase, |
| | | elevated creatine and anorexia requiring an additional 2 |
| | | patients to be added at this dose |
| 3 | 10 | One patient treated with minimal toxicity |
| 4 | 16.5 | One patient treated expired 4 days after beginning cycle due |
| | | to disease process (unrelated to drug study). The patient |
| | | was replaced by another patient which was found to have |
| | | brain metastasis requiring radiation therapy and was |
| | | replaced. The next patient showed Grade 2 toxicities |
| | | (anemia, hypertension, tachycardia, diaphoresis, flushing |
| | | and fatigue) requiring 2 more patients at this dose. These |
| | | two patients had minimal toxicities. |
| 5 | 25 | Six patients were treated with 2 having DLTs in the form of |
| | | grade 4 constipation, Grade 3 tumor pain, nausea, vomiting, |
| | | anemia and Grade 4 neutropenia (1 patient). |
| 6 | 30 | One patient treated developed Grade 3 leukopenia. |
| 7* | 21 | Seven patients enrolled but only 6 were evaluable for |
| | | toxicity. One patient was replaced since treatment was |
| | | incomplete and non-evaluable. At this dose level, one |
| | | patient exhibited Grade 3 nausea and vomiting. This does |
| | | was determined to be the MTD and the study was closed. |

*Since minimal toxicity was seen at 16.5 mg/m² dose level and the increment from 16.5 to 25 mg/m² represented a 50% increase, it was elected to evaluate an intermediate dose level at 21 mg/m².

TABLE 5
Anhydrovinblastine Pharmacokinetic Parameters

| Dose | | | A | T_{1/2} al | AUC | B | T_{1/2} be | CL | Cₘₐₓ | Vₛₛ |
|---|---|---|---|---|---|---|---|---|---|---|
| (mg/m²) | N | | (ng/mL) | (h) | (ug/mL * h) | (ng/mL) | (h) | (L/h/m²) | (ng/mL) | (L/m²) |
| 2.5 | 1 | Value | 134.71 | 0.12 | 71.64 | 5.03 | 6.63 | 34.90 | 28.23 | 226.04 |
| 5 | 3 | Mean | 281.82 | 0.14 | 571.75 | 8.04 | 44.36 | 10.61 | 51.37 | 534.05 |
| | | Min | 135.55 | 0.07 | 282.20 | 6.27 | 27.54 | 6.30 | 38.65 | 445.34 |
| | | Median | 178.39 | 0.17 | 639.67 | 8.11 | 42.41 | 7.82 | 53.07 | 534.71 |
| | | Max | 531.52 | 0.17 | 793.39 | 9.73 | 63.13 | 17.72 | 62.40 | 622.10 |
| | | CV % | 77.11 | 41.87 | 45.87 | 21.53 | 40.30 | 58.42 | 23.28 | 16.55 |
| 10 | 1 | Value | 109.85 | 0.25 | 247.19 | 11.49 | 12.50 | 40.45 | 48.57 | 614.22 |
| 16.5 | 5 | Mean | 509.47 | 0.22 | 824.58 | 22.50 | 21.35 | 25.13 | 163.03 | 486.18 |
| | | Min | 263.49 | 0.13 | 511.26 | 6.64 | 8.21 | 9.21 | 96.35 | 298.06 |
| | | Median | 478.82 | 0.20 | 541.21 | 21.87 | 21.82 | 30.49 | 171.48 | 485.95 |
| | | Max | 816.42 | 0.39 | 1791.22 | 34.16 | 33.52 | 32.27 | 231.52 | 625.73 |
| | | CV % | 41.58 | 46.33 | 66.79 | 51.82 | 47.25 | 36.55 | 30.17 | 28.34 |
| 21 | 3 | Mean | 774.37 | 0.26 | 1094.91 | 25.92 | 22.88 | 19.87 | 257.21 | 488.51 |
| | | Min | 322.90 | 0.19 | 918.58 | 24.50 | 19.00 | 14.94 | 179.07 | 471.27 |
| | | Median | 802.43 | 0.20 | 964.03 | 24.72 | 19.15 | 21.78 | 252.10 | 486.76 |
| | | Max | 1197.78 | 0.39 | 1402.11 | 28.54 | 30.50 | 22.86 | 340.46 | 507.51 |
| | | CV % | 56.58 | 43.26 | 24.39 | 8.76 | 28.83 | 21.51 | 31.42 | 3.72 |
| 25 | 6 | Mean | 1122.91 | 0.18 | 764.83 | 34.51 | 10.68 | 36.43 | 273.43 | 380.18 |
| | | Min | 379.79 | 0.11 | 436.85 | 20.78 | 6.73 | 20.80 | 108.52 | 227.92 |
| | | Median | 1062.6 | 0.16 | 750.85 | 35.06 | 10.54 | 33.48 | 287.71 | 270.11 |
| | | Max | 1952.78 | 0.36 | 1202.01 | 48.76 | 16.29 | 57.23 | 368.79 | 762.66 |
| | | CV % | 59.00 | 49.42 | 35.74 | 32.63 | 33.27 | 36.30 | 34.30 | 55.41 |
| 30 | 1 | Value | 3559.02 | 0.10 | 1797.92 | 36.71 | 23.83 | 16.69 | 571.08 | 403.55 |
| All Data | 20 | Mean | | 0.19 | | | 20.8 | 26.4 | | 451 |
| | | Min | | 0.07 | | | 6.63 | 6.30 | | 226 |
| | | Median | | 0.17 | | | 17.60 | 25.30 | | 479 |
| | | Max | | 0.393 | | | 63.10 | 57.30 | | 763 |
| | | CV % | | 47 | | | 68 | 49 | | 34 |

We claim:

1. A method of treating cancer in a mammal comprising administering to said mammal an effective amount of 3′,4′-anhydrovinblastine or a pharmaceutically acceptable salt thereof, wherein said cancer is pancreatic cancer, neuroendocrine cancer or soft tissue sarcoma.

2. The method according to claim 1, wherein said mammal is a human.

3. A method of treating an advanced cancer in a mammal comprising administering to said mammal an effective amount of 3′,4′-anhydrovinblastine or a pharmaceutically acceptable salt thereof.

4. The method according to claim 3, wherein said mammal is a human.

5. The method according to claim 3, wherein said advanced cancer is a solid tumor.

6. The method according to claim 3, wherein said advanced cancer is pancreatic cancer, neuroendocrine cancer or soft tissue sarcoma.

7. The method according to claim 3, wherein said advanced cancer is lung cancer, colorectal cancer or breast cancer.

8. The method according to claim 7, wherein said lung cancer is non-small cell lung carcinoma.

9. The method according to claim 3, wherein said advanced cancer is a metastatic cancer.

10. The method according to claim 9, wherein said metastatic cancer is metastatic soft tissue sarcoma or metastatic neuroendocrine cancer.

11. The method according to claim 9, wherein said metastatic cancer is metastatic lung cancer.

12. The method according to claim 11, wherein said metastatic lung cancer is metastatic non-small cell lung carcinoma.

13. The method according to claim 3, wherein said advanced cancer is a refractory cancer.

14. The method according to claim 13, wherein said refractory cancer is pancreatic cancer, neuroendocrine cancer or soft tissue sarcoma.

15. The method according to claim 13, wherein said refractory cancer is lung cancer, colorectal cancer or breast cancer.

16. The method according to claim 15, wherein said lung cancer is non-small cell lung carcinoma.

17. The method according to claim 3, wherein said cancer is an adenocarcinoma.

18. The method according to claim 17, wherein said adenocarcinoma is pancreatic cancer or prostate cancer.

19. The method according to claim 17, wherein said adenocarcinoma is breast cancer or lung cancer.

20. A method of treating cancer in a mammal comprising administering to said mammal a dose of between about 2 and about 30 mg of 3′,4′-anhydrovinblastine (AHVB)/m², or a pharmaceutically acceptable salt thereof.

21. The method according to claim 20, wherein said dose is between about 10 and about 21 mg AHVB/m².

22. The method according to claim 20, wherein said mammal is a human.

23. The method according to claim 20, wherein said cancer is a solid tumor.

24. The method according to claim 20, wherein said cancer is pancreatic cancer, neuroendocrine cancer or soft tissue sarcoma.

25. The method according to claim 20, wherein said cancer is lung cancer, colorectal cancer or breast cancer.

26. The method according to claim 25, wherein said lung cancer is non-small cell lung carcinoma.

27. The method according to claim 20, wherein said cancer is an advanced cancer.

28. The method according to claim 27, wherein said advanced cancer is pancreatic cancer, neuroendocrine cancer or soft tissue sarcoma.

29. The method according to claim 27, wherein said cancer is lung cancer, colorectal cancer or breast cancer.

30. The method according to claim 29, wherein said lung cancer is non-small cell lung carcinoma.

31. The method according to claim 20, wherein said cancer is a metastatic cancer.

32. The method according to claim 31, wherein said metastatic cancer is metastatic soft tissue sarcoma or metastatic neuroendocrine cancer.

33. The method according to claim 31, wherein said metastatic cancer is metastatic lung cancer.

34. The method according to claim 33, wherein said metastatic cancer is metastatic non-small cell lung carcinoma.

35. The method according to claim 20, wherein said cancer is a refractory cancer.

36. The method according to claim 35, wherein said refractory cancer is pancreatic cancer, neuroendocrine cancer or soft tissue sarcoma.

37. The method according to claim 35, wherein said refractory cancer is lung cancer, colorectal cancer or breast cancer.

38. The method according to claim 37, wherein said lung cancer is non-small cell lung carcinoma.

39. The method according to claim 20, wherein said cancer is an adenocarcinoma.

40. The method according to claim 39, wherein said adenocarcinoma is pancreatic cancer or prostate cancer.

41. The method according to claim 39, wherein said adenocarcinoma is breast cancer or lung cancer.

42. The method according to claim 41, wherein said lung cancer is non-small cell lung carcinoma.

43. A pharmaceutical composition comprising 3′,4′-anhydrovinblastine (AHVB) and one or more pharmaceutically acceptable, inert or physiologically active carriers, diluents or adjuvants, said AHVB being formulated for administration to a mammal at a dose of between about 2 and about 30 mg AHVB/m².

44. The pharmaceutical composition according to claim 43, wherein said AHVB is formulated for administration to a mammal at a dose of between about 10 and about 21 mg AHVB/m².

45. The pharmaceutical composition according to claim 43, wherein said mammal is a human.
